(19) United States
(12) Patent Application Publication
(10) Pub. No.: US 2010/0105727 A1
(43) Pub. Date: Apr. 29, 2010

(54) NOVEL BENZAMIDE DERIVATIVES AND PROCESS FOR THE PREPARTION THEREOF

(75) Inventors: Moo-Hi Yoo, Seoul (KR); Jae-Keol Rhee, Suwon-si (KR); Weon-Bin Im, Yongin-si (KR); Sung-Hak Choi, Seongnam-si (KR); Eun-Jung Kim, Seoul (KR); Jung-Sang Park, Suwon-si (KR); Sun-Ho Choi, Seoul (KR); Tae-Kyoung Shon, Seoul (KR); Hyun-Jung Sung, Suwon-si (KR); Ja-Young Kim, Seoul (KR); Ju-Hee Shon, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: DONG-A-PHARM. CO., LTD., Seoul (KR)

(21) Appl. No.: 12/531,677

(22) PCT Filed: Mar. 14, 2008
(86) PCT No.: PCT/KR2008/001465

§ 371 Date: Jan. 8, 2010

(30) Foreign Application Priority Data
Mar. 16, 2007 (KR) 10-2007-0025952

(51) Int. Cl. A61K 31/47; C07D 401/02; A61K 31/4545; A61K 31/454; C07D 405/02; A61K 31/4525; C07D 215/00; A61P 1/00; A61P 25/00; A61P 9/00; A61P 3/10

(52) U.S. Cl. 514/311; 546/188; 514/316; 546/210; 514/326; 546/208; 546/196; 514/320; 546/152; 546/193; 514/318

(57) ABSTRACT

The present invention provides a novel benzamide derivative represented by formula 1 and an isomer, a pharmaceutically acceptable salt or hydrate thereof, and a composition for activating a 5-HT<sb>4</sb> receptor comprising the same, as an active ingredient. Benzamide derivatives of the present invention has superior affinity for 5-HT<sb>4</sb> receptors, capability to reduce the gastric evacuation time, capability to alleviate ventricular tachycardia, ventricular fibrillation, torsades de pointes and QT prolongation, and low toxicity. Therefore, benzamide derivatives of the present invention are therapeutically effective for digestive system diseases.

### TECHNICAL FIELD

The present invention relates to a novel benzamide derivative represented by formula 1 and an isomer, a pharmaceutically acceptable salt or a hydrate thereof, and a composition for activating a 5-HT₄ receptor comprising the same, as an active ingredient.

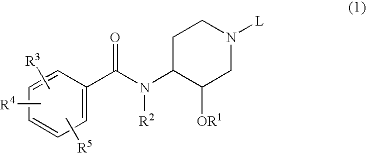

### BACKGROUND ART

It is generally known that 5-HT₄ receptor agonists are therapeutically effective for the treatment of various disease conditions, such as gastroesophageal reflux disease, gastrointestinal diseases, gastric motility disorders, non-ulcer dyspepsia, functional dyspepsia, irritable bowel syndrome (IBS), constipation, post-operative ileus, gastroparesis, dyspepsia, esophagitis, gastroesophageal diseases, motion sickness, central nervous system diseases, Alzheimer's disease, cognitive impairment, emesis, migraine, neurological diseases, pain, cardiovascular diseases, heart failure, cardiac arrhythmia, diabetes and apnea syndrome (see Tips, 1992, 13, 141; Ford A. P. D. W. et al., Med. Res. Rev., 1993, 13. 633; Gullikson G. W. et al., Drug Dev. Res., 1992, 26, 405; Richard M. Eglen et al., Tips, 1995, 16, 391; Bockaert J. et al., CNS Drugs, 1, 6; Romanelli M. N. et al., Arzheim Forsch./Drug Res., 1993, 43, 913; Kaumann A. et al., Naunyn-Schmiedeberg's. 1991, 344, 150; and Romanelli M. N. et al., Arzheim Forsch./Drug Res., 1993, 43, 913).

A typical 5-HT₄ receptor agonist cisapride is one of a class of compounds known as benzamide derivatives, the parent compound of which is metoclopramide. U.S. Pat. Nos. 4,962,115, 5,057,525 and 5,137,896 disclose N-(3-hydroxy-4-piperidinyl)benzamides including cisapride. These compounds are known to stimulate gastrointestinal motility.

Benzamide derivatives have several prominent pharmacological actions. These excellent pharmacological activities of the benzamide derivatives are due to their effects on the nervous systems which are regulated by the neurotransmitter serotonin. The role of serotonin, that is, the pharmacological action of benzamide derivatives has been broadly implicated in a variety of conditions for many years. Thus, a great deal of study and research has focused on locating the production and storage sites of serotonin as well as the location of serotonin receptors in the human body in order to determine the relationship between these sites and various disease states or conditions.

Another noticeable action of the benzamide derivatives is in augmenting gastrointestinal smooth muscle activity from the esophagus through the proximal small bowel, thus accelerating esophageal and small intestinal transit as well as facilitating gastric emptying and increasing lower esophageal sphincter tone (Decktor et al., Eur. J. Pharmacol. 147: 313-316, 1988). Although the benzamide derivatives are not cholinergic receptor agonists per se, the aforementioned smooth muscle effects may be blocked by muscarinic receptor blocking agents such as atropine, or by neurotransmission inhibitors of the tetrodotoxin type which affect sodium channels. Further, similar blocking activity has been reported for the contractile effects of serotonin in the small intestine. It is believed that the primary smooth muscle effects of the benzamide derivatives are the results of agonistic action on serotonin receptors referred to as 5-HT₄ receptors which are located on interneurons in the myenteric plexus of the gut wall. Activation of these receptors subsequently enhances the release of acetylcholine from parasympathetic nerve endings located near surrounding smooth muscle fibers, and it is the combination of acetylcholine with its receptors on smooth muscle membranes, which is the actual trigger for muscle contraction.

Cisapride is used primarily for the treatment of gastroesophageal reflux disease (GERD) which is characterized by the backward flow of the stomach contents into the esophagus. One of the most important factors in the pathogenesis of gastroesophageal reflux disease is a reduction in the pressure barrier due to the failure of the lower esophageal sphincter. Dysfunction of the lower esophageal sphincter may arise due to a low basal pressure or sphincter relaxation, or due to a non-compensated increase in the intragastric pressure. Other factors responsible for the pathogenesis of the disease are delayed gastric emptying, insufficient esophageal clearing due to impaired peristalsis and the corrosive nature of the reflux materials which may damage the esophageal mucosa. Cisapride is thought to increase the lower esophageal sphincter pressure and improve esophageal transit.

Because of its activity as a gastrointestinal prokinetic agent, cisapride may be effective for the treatment of dyspepsia, gastroparesis, constipation, post-operative ileus, intestinal pseudo-obstruction, and the like. The term “dyspepsia” as used herein means a condition characterized by an impairment of the power or function of digestion that may arise as a symptom of a primary gastrointestinal dysfunction or as a complication due to other disorders such as appendicitis, gallbladder disturbances, or malnutrition. The term “gastroparesis” as used herein means a paralysis of the stomach brought about by a motor abnormality in the stomach or as a complication of diseases such as diabetes, progressive systemic sclerosis, anorexia nervosa, or myotonic dystrophy. The term “constipation” as used herein means a condition characterized by infrequent or difficult evacuation of feces resulting from conditions such as lack of intestinal muscle tone or intestinal spasticity. The term “post-operative ileus” as used herein means an obstruction in the intestine due to a disruption in muscle tone following surgery. The term “intestinal pseudo-obstruction” as used herein means a condition characterized by constipation, colicky pain, and vomiting, but without evidence of physical obstruction.

More than 90% of a dose of cisapride is metabolized mainly by oxidative N-dealkylation at the piperidine nitrogen or by aromatic hydroxylation occurring on either the 4-fluorophenoxy or benzamide rings. The administration of cisapride to a human has been found to cause serious adverse side effects including CNS disorders, increased systolic pressure, interactions with other drugs, diarrhea, and abdominal cramping. Further, it has been reported that intravenous administration of cisapride demonstrates the occurrence of additional adverse side effects not experienced after oral administration of cisapride (Stacher et al., 1987 Digestive Diseases and Sciences 31 (11): 1223-1230).

It is believed that these side effects are caused by the metabolites which result from the oxidative dealkylation or aromatic hydroxylation of the compound which occurs in the cytochrome P450 detoxification system. Further, cisapride exhibits susceptibility to a variety of undesirable drugs or drug interactions, which is also attributable to metabolism by the cytochrome P450 system.

Between July 1993 and December 1999, cisapride (PREPULSID™, Janssen Pharmaceutica Products, L.P.) has been reportedly associated with at least 341 serious cardiac arrhythmias. These arrhythmias include ventricular tachycardia, ventricular fibrillation, torsades de pointes (ventricular arrhythmia), and prolongation of the QT interval. Eighty deaths have been reported. Janssen Pharmaceutica has stopped marketing cisapride in the United States due to the risk of such adverse effects. It is only available through an investigational limited access program.

Certain cisapride derivatives have been disclosed in U.S. Pat. No. 6,552,046 and WO01/093849. However, the safety of 5-HT₄ receptor agonists with gastrointestinal prokinetic activity has been limited due to undesirable cardiac effects (prolongation of QT intervals, tachycardia, and torsades de pointes) and adverse drug interactions due to hepatic cytochrome P450 metabolism. Therefore, there has been a need for development of a GI prokinetic agent that lacks these liabilities in therapeutic areas including GERD and gastric emptying disorders.

As a result of a variety of extensive and intensive studies and experiments to solve the problems as described above, the inventors of the present invention succeeded in synthesis of novel benzamide derivatives which exhibit agonistic activity via strong binding with a 5-HT₄ receptor and good gastrointestinal absorption and which are capable of minimizing adverse side effects of cardiac arrhythmia exhibited by cisapride. The present invention has been completed based on this finding.

### DISCLOSURE OF THE INVENTION

### Technical Problem

Therefore, it is an object of the present invention to provide a novel benzamide derivative and a process for preparing the same.

It is another object of the present invention to provide a composition for activating a 5-HT₄ receptor, comprising a novel benzamide derivative capable of minimizing the side effect of cardiac arrhythmia, as an active ingredient, and a use and a preparation thereof.

### Technical Solution

In accordance with an aspect of the present invention, the above and other objects can be accomplished by the provision of a novel benzamide derivative represented by formula 1:

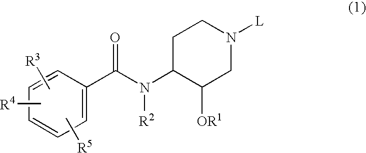

wherein:

R¹ is hydrogen or C₁₋₆ alkyl;

R² is hydrogen or C₁₋₆ alkyl;

R³, R⁴ and R⁵ are independently hydrogen, C₁₋₆ alkyl, C₁₋₆ alkoxy, amino, hydroxy, cyano, nitro, or halogen; and

L is

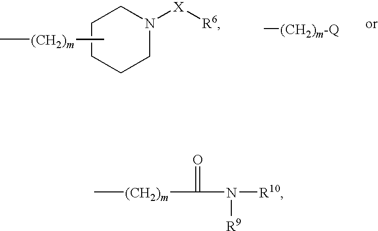

wherein m is an integer of 1 to 5; X is —(C═O)—, —(C═S)—, or —SO₂—; R⁶ is C₁₋₁₀ alkyl, C₁₋₁₀ alkenyl, C₁₋₁₀ alkoxy, C₁₋₁₀ thioalkoxy, or NR⁷R⁸ wherein R⁷ and R⁸, which are identical or different, are independently hydrogen or C₁₋₁₀ alkyl; Q is pyrrole, 1,2,3-triazole, 1,2,4-triazole, tetrazole, oxazole, isoxazole, thiazole, thiadiazole, oxadiazole, or benzofuran, each of which being optionally substituted by C₃₋₁₀ cycloalkyl, C₁₋₆ alkyl or nitro; and R⁹ and R¹⁰, which are identical or different, are independently pyridine, indole, or quinoline, each of which being optionally substituted by hydrogen or C₁₋₆ alkyl.

The benzamide derivative of formula 1 in accordance with the present invention may be used in the form of a pharmaceutically acceptable salt thereof. The salt may be an acid addition salt with an acceptable free acid. The free acid may be inorganic or organic acid. Examples of the inorganic acid may include hydrochloric acid, hydrobromic acid, sulfuric acid, phosphoric acid, and the like. Examples of the organic acid may include citric acid, acetic acid, lactic acid, maleic acid, umaric acid, gluconic acid, methanesulfonic acid, glycolic acid, succinic acid, 4-toluenesulfonic acid, trifluoroacetic acid, galacturonic acid, embonic acid, glutamic acid, aspartic acid and the like.

An acid addition salt of a free base of a compound having formula 1 (including racemic mixture and optically active forms) may be prepared using a conventional method known in the art, for example by mixing a free base of the compound of formula 1 with a certain acid in a suitable solvent, which is then followed by evaporation to form a salt or addition of a non-solvent to precipitate a salt. For example, mention may be made of a method which involves treating a solution or suspension of a free base in a non-reactive solvent with a certain acid, followed by concentration under reduced pressure, crystallization, or any standard chemical manipulation to form a desired salt.

As will be apparent to those skilled in the art, a compound represented by formula 1 has one or more asymmetric carbon atoms, and therefore may be present in the form of an optically active isomer or racemic mixture, all of which fall within the scope of the present invention. Racemic resolution for producing optically active isomers of a compound represented by formula 1 may be carried out by a conventional resolution method known in the art. For example, a base of the compound of formula 1 is reacted with an optically active acid to form a salt thereof, from which dextro (right) and levo (left) forms of optical isomers are then separated by fractional crystallization. Examples of acids suitable for dissolution of the compound of formula 1 may include optically active forms of tartaric acid, ditolyltartaric acid, dibenzoyltartaric acid, malic acid, mandelic acid and camphorsulfonic acid and any optically active acid known in the related art. Preferably, more biologically and optically active stereoisomeric forms of a compound of formula 1 are preferably separated.

Further, compounds of formula 1, and isomers or pharmaceutically acceptable salts thereof may exhibit polymorphism. These compounds may be present in the form of tautomers or solvates (e.g., hydrates, etc).

Unless otherwise indicated, the terms defined herein shall have the meanings as follows.

As used herein, the term “alkyl” refers to a linear or branched-chain monovalent saturated C₁-C₂₀ hydrocarbon radical containing only carbon and hydrogen atoms. Examples of the alkyl radical may include methyl, ethyl, propyl, isopropyl, 2,2-dimethylpropyl, butyl, isobutyl, sec-butyl, tert-butyl, 3-methylbutyl, pentyl, 3-methylpentyl, 4-methylpentyl, n-hexyl, 2-ethylhexyl, octyl, dodecyl, and the like.

As used herein, the term “alkenyl” refers to a linear or branched-chain divalent saturated C₁-C₂₀ hydrocarbon radical containing only carbon and hydrogen atoms. Examples of the alkenyl radical may include ethenyl, 1-propenyl, 2-propenyl, isopropenyl, butenyl, isobutenyl, pentenyl, n-hexenyl, ocenyl, dodecenyl, and the like.

As used herein, the term “alkoxy” refers to a radical OR wherein R is alkyl or alkenyl as defined above. Examples of the alkoxy radical may include methoxy, ethoxy, propoxy, isopropoxy, butoxy, isobutoxy, sec-butoxy, tert-butoxy, pentoxy, 3-methylpentoxy, 4-methylpentoxy, n-hexoxy, 2-ethylhexoxy, and the like.

As used herein, the term “thioalkoxy” refers to a radical SR wherein R is alkyl as defined above. Examples of the thioalkoxy radical may include thiomethoxy, thioethoxy, thiopropoxy, thioisopropoxy, thiobutoxy, thioisobutoxy, sec-thiobutoxy, tert-thiobutoxy, thiopentoxy, thiohexoxy, and the like.

As used herein, the term “cycloalkyl” refers to a monovalent saturated hydrocarbon cyclic radical consisting of one or more rings which may be optionally substituted by hydroxy, cyano, alkyl, alkoxy, halogen, nitro, alkoxycarbonyl, amino, dialkylamino, aminocarbonyl or carbonylamino. Examples of the cycloalkyl radical may include cyclopropyl, cyclobutyl, cyclopentyl, cyclohexyl, cycloheptyl, bicyclo[2.2.1]heptyl, bicyclo[3.2.1]octyl, and the like.

As used herein, the term “halogen” refers to a fluoro, bromo, chloro or iodo radical.

As used herein, the term “treating” refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

Specifically, preferred examples of novel benzamide derivatives in accordance with the present invention may include the following compounds:
(1) ethyl 4-[(cis-4-(4-amino-5-chloro-2-methoxybenzamido)-3-methoxypiperidin-1-yl)methyl]piperidine-1-carboxylate,
(2) ethyl 4-[((3S,4R)-4-(4-amino-5-chloro-2-methoxybenzamido)-3-methoxypiperidin-1-yl)methyl]piperidine-1-carboxylate,
(3) ethyl 4-[2-(cis-4-(4-amino-5-chloro-2-methoxybenzamido)-3-methoxypiperidin-1-yl)ethyl]piperidine-1-carboxylate,
(4) ethyl 2-[2-(cis-4-(4-amino-5-chloro-2-methoxybenzamido)-3-methoxypiperidin-1-yl)ethyl]piperidine-1-carboxylate,
(5) methyl 4-[(cis-4-(4-amino-5-chloro-2-methoxybenzamido)-3-methoxypiperidin-1-yl)methyl]piperidine-1-carboxylate,
(6) propyl 4-[(cis-4-(4-amino-5-chloro-2-methoxybenzamido)-3-methoxypiperidin-1-yl)methyl]piperidine-1-carboxylate,
(7) butyl 4-[(cis-4-(4-amino-5-chloro-2-methoxybenzamido)-3-methoxypiperidin-1-yl)methyl]piperidine-1-carboxylate,
(8) isopropyl 4-[(cis-4-(4-amino-5-chloro-2-methoxybenzamido)-3-methoxypiperidin-1-yl)methyl]piperidine-1-carboxylate,
(9) isobutyl 4-[(cis-4-(4-amino-5-chloro-2-methoxybenzamido)-3-methoxypiperidin-1-yl)methyl]piperidine-1-carboxylate,
(10) allyl 4-[(cis-4-(4-amino-5-chloro-2-methoxybenzamido)-3-methoxypiperidin-1-yl)methyl]piperidine-1-carboxylate,
(11) 2-ethylhexyl 4-[(cis-4-(4-amino-5-chloro-2-methoxybenzamido)-3-methoxypiperidin-1-yl)methyl]piperidine-1-carboxylate,
(12) 3-methyl-pentyl 4-[(cis-4-(4-amino-5-chloro-2-methoxybenzamido)-3-methoxypiperidin-1-yl)methyl]piperidine-1-carboxylate,
(13) 4-methyl-pentyl 4-[(cis-4-(4-amino-5-chloro-2-methoxybenzamido)-3-methoxypiperidin-1-yl)methyl]piperidine-1-carboxylate,
(14) cis-4-amino-5-chloro-N-[1-((1-isobutyrylpiperidin-4-yl)methyl)-3-methoxypiperidin-4-yl]-2-methoxybenzamide,
(15) (3S,4R)-4-amino-5-chloro-N-[1-((1-isobutyrylpiperklin-4-yl)methyl)-3-methoxypiperidin-4-yl]-2-methoxybenzamide,
(16) cis-4-amino-5-chloro-N-[1-(2-(1-isobutyrylpiperidin-4-yl)ethyl)-3-methoxypiperidin-4-yl]-2-methoxybenzamide,
(17) cis-4-amino-5-chloro-N-[1-((1-propionylpiperidin-4-yl)methyl)-3-methoxypiperidin-4-yl]-2-methoxybenzamide,
(18) cis-4-amino-5-chloro-N-[1-(2-(1-propionylpiperidin-4-yl)ethyl)-3-methoxypiperidin-4-yl]-2-methoxybenzamide,
(19) cis-4-amino-5-chloro-N-[1-((1-propionylpiperidin-3-yl)methyl)-3-methoxypiperidin-4-yl]-2-methoxybenzamide,
(20) cis-4-amino-5-chloro-N-[1-((1-butyrylpiperidin-4-yl)methyl)-3-methoxypiperidin-4-yl]-2-methoxybenzamide,
(21) cis-4-amino-5-chloro-N-[1-((1-butyrylpiperidin-3-yl)methyl)-3-methoxypiperidin-4-yl]-2-methoxybenzamide,
(22) cis-4-amino-5-chloro-N-[1-((1-pentanoylpiperidin-4-yl)methyl)-3-methoxypiperidin-4-yl]-2-methoxybenzamide,
(23) cis-4-amino-5-chloro-N-[1-((1-hexanoylpiperidin-4-yl)methyl)-3-methoxypiperidin-4-yl]-2-methoxybenzamide,
(24) cis-4-amino-5-chloro-N-[1-((1-(2-methylpentanoyl)piperidin-4-yl)methyl)-3-methoxypiperidin-4-yl]-2-methoxybenzamide,
(25) cis-4-amino-5-chloro-N-[1-((1-(3-methylbutanoyl)piperidin-4-yl)methyl)-3-methoxypiperidin-4-yl]-2-methoxybenzamide,
(26) cis-4-amino-5-chloro-N-[1-((1-(3,3-dimethylbutanoyl)piperidin-4-yl)methyl)-3-methoxypiperidin-4-yl]-2-methoxybenzamide,
(27) cis-4-amino-5-chloro-N-[1-((1-(4-methylpentanoyl)piperidin-4-yl)methyl)-3-methoxypiperidin-4-yl]-2-methoxybenzamide,
(28) cis-4-amino-5-chloro-N-[1-((1-acetylpiperidin-4-yl)methyl)-3-methoxypiperidin-4-yl]-2-methoxybenzamide,
(29) cis-4-amino-5-chloro-N-[1-((1-pivaloylpiperidin-4-yl)methyl)-3-methoxypiperidin-4-yl]-2-methoxybenzamide,
(30) cis-4-amino-5-chloro-N-[1-((1-(2-methylpropanethioyl)piperidin-4-yl)methyl)-3-methoxypiperidin-4-yl]-2-methoxybenzamide,
(31) cis-4-amino-5-chloro-N-[1-((1-ethanethioylpiperidin-4-yl)methyl)-3-methoxypiperidin-4-yl]-2-methoxybenzamide,
(32) cis-4-amino-5-chloro-N-[1-((1-propanethioylpiperidin-4-yl)methyl)-3-methoxypiperidin-4-yl]-2-methoxybenzamide,
(33) cis-4-amino-5-chloro-N-[1-((1-pentanethioylpiperidin-4-yl)methyl)-3-methoxypiperidin-4-yl]-2-methoxybenzamide,
(34) cis-4-amino-5-chloro-N-[1-((1-hexanethioylpiperidin-4-yl)methyl)-3-methoxypiperidin-4-yl]-2-methoxybenzamide,
(35) cis-4-amino-5-chloro-N-[1-((1-butanethioylpiperidin-4-yl)methyl)-3-methoxypiperidin-4-yl]-2-methoxybenzamide,
(36) cis-4-amino-5-chloro-N-[1-((1-(3-methylbutanethioyl)piperidin-4-yl)methyl)-3-methoxypiperidin-4-yl]-2-methoxybenzamide,
(37) cis-4-amino-5-chloro-N-[1-((1-(4-methylpentanethioyl)piperidin-4-yl)methyl)-3-methoxypiperidin-4-yl]-2-methoxybenzamide,
(38) cis-4-amino-5-chloro-N-[1-((1-(2,2-dimethylpropanethioyl)piperidin-4-yl)methyl)-3-methoxypiperidin-4-yl]-2-methoxybenzamide,
(39) S-ethyl 4-[(cis-4-(4-amino-5-chloro-2-methoxybenzamido)-3-methoxypiperidin-1-yl)methyl]piperidine-1-carbothioate,
(40) S-propyl 4-[(cis-4-(4-amino-5-chloro-2-methoxybenzamido)-3-methoxypiperidin-1-yl)methyl]piperidine-1-carbothioate,
(41) S-butyl 4-[(cis-4-(4-amino-5-chloro-2-methoxybenzamido)-3-methoxypiperidin-1-yl)methyl]piperidine-1-carbothioate,
(42) cis-4-amino-5-chloro-N-[1-((1-(isopropylsulfonyl)piperidin-4-yl)methyl)-3-methoxypiperidin-4-yl]-2-methoxybenzamide,
(43) cis-4-amino-5-chloro-N-[1-((1-(methylsulfonyl)piperidin-4-yl)methyl)-3-methoxypiperidin-4-yl]-2-methoxybenzamide,
(44) (3S,4R)-4-amino-5-chloro-N-[1-((1-(methylsulfonyl)piperidin-4-yl)methyl)-3-methoxypiperidin-4-yl]-2-methoxybenzamide,
(45) cis-4-amino-5-chloro-N-[1-(2-(1-(methylsulfonyl)piperidin-4-yl(ethyl)-3-methoxypiperidin-4-yl]-2-methoxybenzamide,
(46) cis-4-amino-5-chloro-N-[1-(3-(1H-1,2,4-triazol-1-yl)propyl)-3-methoxypiperidin-4-yl]-2-methoxybenzamide,
(47) (3S,4R)-4-amino-5-chloro-N-[1-(3-(1H-1,2,4-triazol-1-yl)propyl)-3-methoxypiperidin-4-yl]-2-methoxybenzamide,
(48) cis-4-amino-5-chloro-N-[1-(3-(1H-tetrazol-2-yl)propyl)-3-methoxypiperidin-4-yl]-2-methoxybenzamide,
(49) cis-4-amino-5-chloro-N-[1-(3-(1H-1,2,3-triazol-1-yl)propyl)-3-methoxypiperidin-4-yl]-2-methoxybenzamide,
(50) cis-4-amino-5-chloro-N-[1-(3-(1H-pyrrol-1-yl)propyl)-3-methoxypiperidin-4-yl]-2-methoxybenzamide,
(51) cis-4-amino-5-chloro-N-[1-(3-(1H-pyrrol-1-yl)ethyl)-3-methoxypiperidin-4-yl]-2-methoxybenzamide,
(52) cis-4-amino-5-chloro-N-[1-(2-(bicyclo[2.2.1]heptan-2-yl)ethyl)-3-methoxypiperidin-4-yl]-2-methoxybenzamide,
(53) cis-4-amino-5-chloro-N-[1-(benzofuran-2-ylmethyl)-3-methoxypiperidin-4-yl]-2-methoxybenzamide,
(54) cis-4-amino-5-chloro-N-[1-(2-(5-methyl-1,2,4-oxadiazol-3-yl)ethyl)-3-methoxypiperidin-4-yl]-2-methoxybenzamide,
(55) (3S,4R)-4-amino-5-chloro-N-[1-(2-(5-methyl-1,2,4-oxadiazol-3-yl)ethyl)-3-methoxypiperidin-4-yl]-2-methoxybenzamide,
(56) cis-4-amino-5-chloro-N-[1-(3-oxo-3-(quinolin-5-ylamino)propyl)-3-methoxypiperidin-4-yl]-2-methoxybenzamide,
(57) cis-4-amino-5-chloro-N-[1-(3-oxo-3-(quinolin-6-ylamino)propyl)-3-methoxypiperidin-4-yl]-2-methoxybenzamide,
(58) cis-4-amino-5-chloro-N-[1-(6-oxo-6-(quinolin-5-ylamino)hexyl)-3-methoxypiperidin-4-yl]-2-methoxybenzamide,
(59) cis-4-amino-5-chloro-N-[1-(3-oxo-3-(4,6-dimethylpyridin-2-ylamino)propyl)-3-methoxypiperidin-4-yl]-2-methoxybenzamide,
(60) cis-4-amino-5-chloro-N-[1-(6-oxo-6-(4,6-dimethylpyridin-2-ylamino)hexyl)-3-methoxypiperidin-4-yl]-2-methoxybenzamide,
(61) cis-4-amino-5-chloro-N-[1-(3-oxo-3-(1H-indol-5-ylamino)propyl)-3-methoxypiperidin-4-yl]-2-methoxybenzamide,
(62) cis-4-amino-5-chloro-N-[1-(6-oxo-6-(1H-indol-5-ylamino)hexyl)-3-methoxypiperidin-4-yl]-2-methoxybenzamide,
(63) 4-[cis-4-(4-amino-5-chloro-2-methoxybenzoylamino)-3-methoxy-piperidin-1-ylmethyl]-piperidine-1-carboxylic acid isopropylamide,
(64) 4-[cis-4-(4-amino-5-chloro-2-methoxybenzoylamino)-3-methoxy-piperidin-1-ylmethyl]-piperidine-1-carboxylic acid dimethylamide,
(65) (3R,4S)-4-amino-5-chloro-N-[1-((1-isobutyrylpiperidin-4-yl)methyl)-3-methoxypiperidin-4-yl]-2-methoxybenzamide,
(66) cis-4-amino-5-chloro-N-[1-((1-isobutyrylpiperidin-4-yl)methyl)-3-methoxypiperidin-4-yl]-2-methoxybenzamide hydrochloride,
(67) cis-4-amino-5-chloro-N-[1-((1-isobutyrylpiperidin-4-yl)methyl)-3-methoxypiperidin-4-yl]-2-methoxybenzamide maleate,
(68) cis-4-amino-5-chloro-N-[1-((1-pivaloylpiperidin-4-yl)methyl)-3-methoxypiperidin-4-yl]-2-methoxybenzamide hydrochloride,
(69) cis-4-amino-5-chloro-N-[1-((1-pivaloylpiperidin-4-yl)methyl)-3-methoxypiperidin-4-yl]-2-methoxybenzamide maleate,
(70) 4-[cis-4-(4-amino-5-chloro-2-methoxybenzoylamino)-3-methoxy-piperidin-1-ylmethyl]-piperidine-1-carboxylic acid isopropylamide hydrochloride, and
(71) 4-[cis-4-(4-amino-5-chloro-2-methoxybenzoylamino)-3-methoxy-piperidin-1-ylmethyl]-piperidine-1-carboxylic acid isopropylamide maleate.

In accordance with another aspect of the present invention, there is provided a process for preparing a benzamide derivative represented by formula 1.

A process for preparing a compound of formula 1-1 which is a compound of formula 1 in accordance with the present invention comprises (1) introducing a substituent at the amine of a compound of formula III to form a compound of formula IV (Step 1); (2) substituting hydroxy of the compound of formula IV by halogen or sulfonate to form a compound of formula V (Step 2); and (3) reacting the resulting compound of formula V with the following piperidine-benzamide compound (a compound of formula II) to prepare a compound of formula 1-1 (Step 3).

Reaction Scheme 1 below illustrates a process for preparing the compound of formula 1-1.

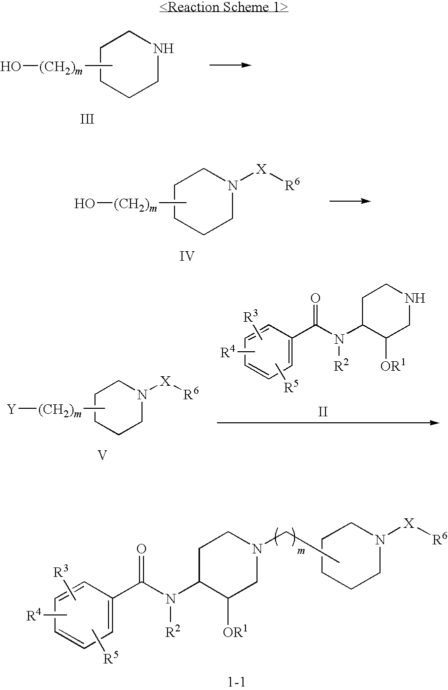

In Reaction Scheme 1, X, R¹, R², R³, R⁴, R⁵, R⁶, and m are as defined in formula 1, an Y is a halogen atom or sulfonate.

The piperidine-benzamide compound (compound of formula II) used in Reaction Scheme 1 may be easily synthesized by a known method (EP 0076530).

Step 1 is intended to introduce a substituent at the amine of a piperidine ring of compound of formula III. The substituent to be introduced herein may be alkyl-, alkoxy- or thioalkoxy-substituted carboxylic acid or carboxylic acid chloride or isocyanate. Preferably, the reaction is initiated at 0° C., followed by gradual elevation to room temperature.

Step 2 includes the substitution of hydroxy of compound of formula IV with halogen or sulfonate. For this purpose, N-bromosuccinimide, carbon tetrabromide or methanesulfonyl chloride may be used. Preferably, halogen may be bromo or chloro. Preferably, the reaction is initiated at 0° C., followed by gradual elevation to room temperature.

Step 3 includes the reaction of compound of formula V with the piperidine-benzamide compound (compound of formula II) to obtain compound of formula 1-1 of the present invention. For this purpose, potassium carbonate and potassium iodide may be used. The reaction solvent may be N,N-dimethylformamide, N,N-dimethylacetamide or ethanol. The reaction is preferably carried out at a temperature of 80 to 90° C.

A process for preparing a compound of formula 1-2 which is a compound of formula 1 in accordance with the present invention comprises (1) substituting Y² of a compound of formula VI with Q to form a compound of formula VII (Step 1); and (2) reacting the resulting compound of formula VII with a piperidine-benzamide compound (a compound of formula II) to prepare a compound of formula 1-2 (Step 2).

Reaction Scheme 2 below illustrates a process for preparing the compound of formula 1-2.

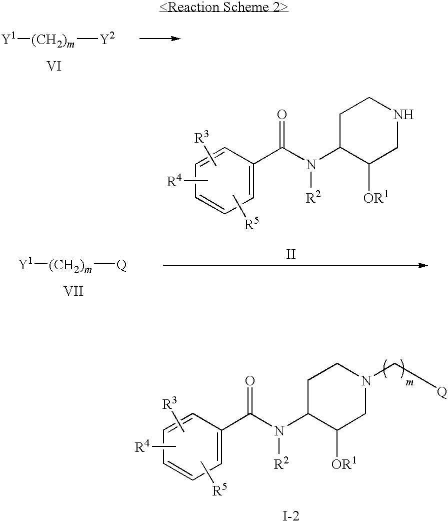

In Reaction Scheme 2, X, R¹, R², R³, R⁴, R⁵, R⁶, m, and Q are as defined in formula 1, and Y¹ and Y², which may be identical or different, are independently a halogen atom.

The piperidine-benzamide compound (Compound of formula II) used in Reaction Scheme 2 may be easily synthesized by any known method in the art.

Step 1 includes the substitution of Y² of compound of formula VI with Q. For this purpose, sodium hydride and N,N-dimethylformamide as a solvent may be, used. Preferably, halogen may be bromo or chloro. Preferably, the reaction is initiated at 0° C., followed by gradual elevation to room temperature.

Step 2 includes the reaction of compound of formula VII with the piperidine-benzamide compound (Compound of formula II) to obtain compound of formula 1-2 of the present invention. For this purpose, potassium carbonate and potassium iodide may be used. The reaction solvent may be N,N-dimethylformamide, N,N-dimethylacetamide or ethanol. The reaction is preferably carried out at a temperature of 80 to 90° C.

Compound of formula VII may be prepared as disclosed in Step 1, or otherwise is easily commercially available.

A process for preparing a compound of formula 1-3 which is a compound of formula 1 in accordance with the present invention comprises (1) reacting an acid chloride compound of formula VIII or XI with an amine compound of formula X to form an amide compound of formula IX or XII (Step 1), and (2) reacting the resulting compound of formula IX or XII with a piperidine-benzamide compound (a compound of formula II) to prepare a compound of formula 1-3 (Step 2).

Reaction Scheme 3 below illustrates a process for preparing the compound of formula 1-3.

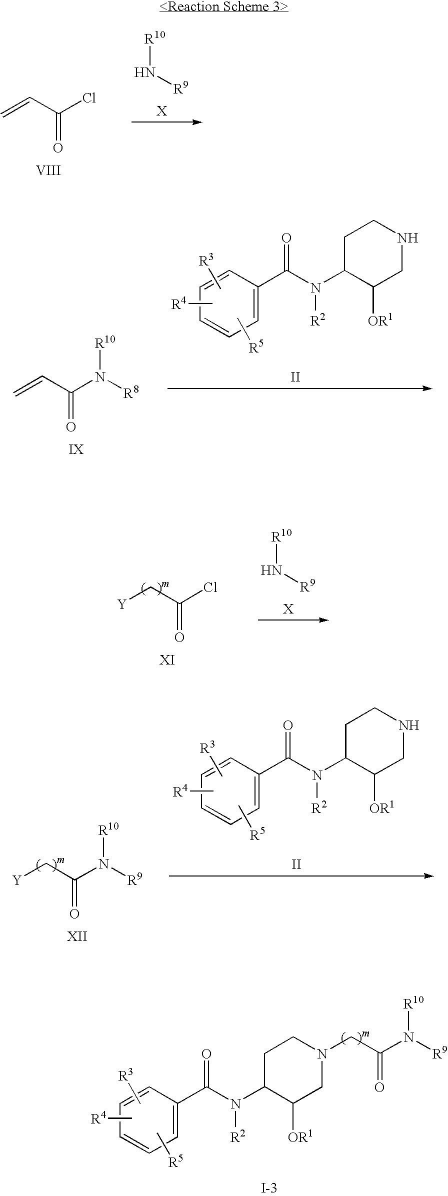

In Reaction Scheme 3, X, R¹, R², R³, R⁴, R⁵, R⁶, R⁹, R¹⁰, and m are as defined in formula 1, and Y is a halogen atom.

The piperidine-benzamide compound (compound of formula II) used in Reaction Scheme 3 may be easily synthesized by any known method in the art.

Step 1 includes the reaction of acryloyl chloride or halogen-substituted acid chloride with amine of formula X to form an amide compound of formula IX or XII. For this purpose, triethylamine and dichloromethane as a solvent may be used. Preferably, halogen may be bromo or chloro. Preferably, the reaction is initiated at 0° C., followed by gradual elevation to room temperature.

Step 2 includes the reaction of compound of formula IX or XII with the piperidine-benzamide compound (compound of formula II) to obtain Compound 1-3 of the present invention. Where appropriate, potassium carbonate and potassium iodide may be used. The reaction solvent may be N,N-dimethylformamide, N,N-dimethylacetamide or ethanol. The reaction is preferably carried out at a room temperature or at a temperature of 80 to 90° C.

In accordance with a further aspect of the present invention, there is provided a 5-HT₄ receptor agonist comprising a benzamide derivative of formula 1 which is capable of minimizing the incidence of cardiac arrhythmia that is a fatal side effect of cisapride, as an active ingredient. The benzamide derivative of formula 1 in accordance with the present invention minimizes the risk of cardiac arrhythmia that is a fatal drug side effect of cisapride and enhances the 5-HT₄ receptor activity, so this compound can be used as a 5-HT₄ receptor agonist.

Further, the present invention provides a composition for activating a 5-HT₄ receptor, comprising a compound of formula 1, or an isomer, a pharmaceutically acceptable salt or a hydrate thereof, as an active ingredient.

The composition for activating a 5-HT₄ receptor in accordance with the present invention may comprise a compound selected from the group consisting of preferred compounds (1) to (71) as listed among the aforesaid novel benzamide derivative compounds.

The composition for activating a 5-HT₄ receptor in accordance with the present invention may be therapeutically effective for the treatment of one or more disease conditions selected from the group consisting of gastroesophageal reflux disease, gastrointestinal diseases, gastric motility disorders, non-ulcer dyspepsia, functional dyspepsia, irritable bowel syndrome (IBS), constipation, post-operative ileus, gastroparesis, dyspepsia, esophagitis, gastroesophageal diseases, motion sickness, central nervous system diseases, Alzheimer's disease, cognitive impairment, emesis, migraine, neurological diseases, pain, cardiovascular diseases, heart failure, cardiac arrhythmia, diabetes and apnea syndrome. That is, the composition of the present invention can be used for the treatment of disease conditions mediated by 5-HT₄ receptor activity, such as gastroesophageal reflux disease, gastrointestinal diseases, gastric motility disorders, non-ulcer dyspepsia, functional dyspepsia, irritable bowel syndrome (IBS), constipation, post-operative ileus, gastroparesis, dyspepsia, esophagitis, gastroesophageal diseases, motion sickness, central nervous system diseases, Alzheimer's disease, cognitive impairment, emesis, migraine, neurological diseases, pain, cardiovascular diseases, heart failure, cardiac arrhythmia, diabetes and apnea syndrome.

Further, the present invention provides a method for activating a 5-HT₄ receptor, comprising administering the aforesaid composition to a mammalian subject. The disease conditions mediated by 5-HT₄ receptor activity can be treated by administering the composition of the present invention to a mammalian subject which is in need of 5-HT₄ receptor activation.

Further, the present invention provides a use of the aforesaid composition for activating a 5-HT₄ receptor.

Further, the present invention provides a method for treating disease conditions mediated by 5-HT₄ receptor activity, comprising administering a compound of formula 1 or an isomer, a pharmaceutically acceptable salt or a hydrate thereof to a mammalian subject in need thereof.

The disease condition mediated by 5-HT₄ receptor activity may be selected from gastroesophageal reflux disease, gastrointestinal diseases, gastric motility disorders, non-ulcer dyspepsia, functional dyspepsia, irritable bowel syndrome (IBS), constipation, post-operative ileus, gastroparesis, dyspepsia, esophagitis, gastroesophageal diseases, motion sickness, central nervous system diseases, Alzheimer's disease, cognitive impairment, emesis, migraine, neurological diseases, pain, cardiovascular diseases, heart failure, cardiac arrhythmia, diabetes and apnea syndrome.

Further, the present invention provides a use of a compound of formula 1 or an isomer, a pharmaceutically acceptable salt or a hydrate thereof, for the preparation of a medicament for treating disease conditions mediated by 5-HT₄ receptor activity in a mammalian subject.

In this connection, the disease condition mediated by 5-HT₄ receptor activity may be selected from gastroesophageal reflux disease, gastrointestinal diseases, gastric motility disorders, non-ulcer dyspepsia, functional dyspepsia, irritable bowel syndrome (IBS), constipation, post-operative ileus, gastroparesis, dyspepsia, esophagitis, gastroesophageal diseases, motion sickness, central nervous system diseases, Alzheimer's disease, cognitive impairment, emesis, migraine, neurological diseases, pain, cardiovascular diseases, heart failure, cardiac arrhythmia, diabetes and apnea syndrome.

Cisapride exhibits excessively high drug affinity for a hERG receptor, which results in prolongation of the cardiac QT interval, so administration of cisapride is accompanied by adverse side effects such as cardiac arrhythmia.

The benzamide derivative in accordance with the present invention is capable of achieving a decrease in the gastric evacuation time while having excellent affinity for the 5-HT₄ receptor, alleviation of adverse side effects (such as ventricular tachycardia, ventricular fibrillation, torsades de pointes and QT prolongation) that are usually suffered by conventional cisapride drugs, low toxicity and excellent in vivo effects.

The composition of the present invention may further comprise one or more additional active ingredients having the pharmacological action identical or similar to that of the benzamide derivative compound of formula 1 or an isomer, a pharmaceutically acceptable salt or a hydrate thereof. Further, the treatment method of the present invention may further comprise administering one or more additional active ingredients having the pharmacological action identical or similar to that of the benzamide derivative, concurrently or sequentially.

For purpose of desired administration, the composition of the present invention may be formulated into a variety of dosage forms by further inclusion of one or more pharmaceutically acceptable carriers in combination with the above-mentioned active ingredient including the benzamide derivative compound of formula 1 or an isomer, a pharmaceutically acceptable salt or a hydrate thereof. For formulation of the composition of a liquid preparation, a pharmaceutically acceptable carrier which is sterile and biocompatible may be used such as saline, sterile water, Ringer's solution, buffered physiological saline, albumin infusion solution, dextrose solution, maltodextrin solution, glycerol, and ethanol. These materials may be used alone or in any combination thereof. If necessary, other conventional additives may be added such as antioxidants, buffers, bacteriostatic agents, and the like. Further, diluents, dispersants, surfactants, binders and lubricants may be additionally added to the composition to prepare injectable formulations such as aqueous solutions, suspensions, and emulsions, or formulations such as pills, capsules, granules, and tablets. Furthermore, the composition may be preferably formulated into a desired dosage form, depending upon diseases to be treated and ingredients, using any appropriate method known in the art, as disclosed in “Remington's Pharmaceutical Sciences,” (latest edition), Mack Publishing Co., Easton, Pa.

Dosage forms of the composition of the present invention may include granules, powders, coated tablets, tablets, capsules, suppositories, syrups, juice, suspensions, emulsions, drops or injectable liquid formulations and sustained-release formulations of active ingredient(s).

Depending upon desired applications of the benzamide derivative compound of formula 1 of the present invention or an isomer, a pharmaceutically acceptable salt or a hydrate thereof, the composition of the present invention can be administered via a conventional route, for example by parenteral administration (intraperitoneally, intramuscularly, intraarterially, intraperitoneally, intrathoracically, percutaneously, intranasally, locally, rectally, intraocularly, intradermally, or by inhalation) or by per oral administration.

As will be apparent to those skilled in the art, the effective dose of the active ingredient in accordance with the present invention may vary depending upon various factors such as weight, age, sex, health, and dietary habits of patients, administration times and routes, excretion rates, and severity of diseases. The benzamide derivative compound of the present invention may be administered at a dose of 1-1000 μg/kg, preferably about 10-500 μg/kg, and more preferably about 83-167 μg/kg once or several times a day.

According to results of the toxicity test by oral administration of the benzamide derivative of the present invention to mice, a 50% lethal dose (LD₅₀) of the benzamide derivative was 1 g/kg or more, thus representing that the compound of the present invention is safe.

### ADVANTAGEOUS EFFECTS

As discussed hereinbefore, a novel benzamide derivative compound in accordance with the present invention provides various advantages such as excellent affinity for 5-HT₄ receptors, capability to reduce the gastric evacuation time, alleviation of ventricular tachycardia, ventricular fibrillation, torsades de pointes and QT prolongation, and promising applicability as a therapeutic agent for digestive system diseases, due to low toxicity.

### MODE FOR INVENTION

Now, the present invention will be described in more detail with reference to the following Examples. These examples are provided only for illustrating the present invention and should not be construed as limiting the scope and spirit of the present invention.

### Example 1

### Preparation of ethyl 4-[(cis-4-(4-amino-5-chloro-2-methoxybenzamido)-3-methoxypiperidin-1-yl)methyl]piperidine-1-carboxylate

### Step 1: Preparation of ethyl 4-(hydroxymethyl)piperidine-1-carboxylate

15 g of 4-piperidinemethanol was dissolved in dichloromethane, and the solution was cooled to 0° C. Then, 38.4 mL of triethylamine (Et₃N) was added followed by slow addition of 13.7 mL of ethylchloroformate. The reaction mixture was warmed to room temperature, stirred for 3 hours, and extracted with dichloromethane. The extracted organic layer was dried over anhydrous magnesium sulfate (MgSO₄), concentrated under reduced pressure, and purified by column chromatography to afford 12 g (49%) of the title compound.

¹H NMR (CDCl₃): δ 4.23-4.08 (m, 4H), 3.49 (d, J=6.0 Hz, 2H) 2.80-2.68 (m, 2H), 1.76-1.60 (m, 3H), 1.24 (t, J=7.2 Hz, 3H), and 1.20-1.08 (m, 2H)

### Step 2: Preparation of ethyl 4-(bromomethyl)piperidine-1-carboxylate

461 mg of ethyl 4-(hydroxymethyl)piperidine-1-carboxylate was dissolved in dichloromethane, and the solution was cooled to 0° C., to which 710 mg of triphenylphosphine (PPh₃) and 482 mg of N-bromosuccinimide (NBS) were then added. The reaction mixture was warmed to room temperature, stirred for 12 hours, and concentrated under reduced pressure. The residue was purified by column chromatography to afford 453 mg (74%) of the title compound.

¹H NMR (CDCl₃): δ 4.28-4.08 (m, 4H), 3.27 (d, J=6.4 Hz, 2H), 2.80-2.64 (m, 2H), 1.86-1.72 (m, 3H), and 1.25-1.12 (m, 5H)

### Step 3: Preparation of ethyl 4-[(cis-4-(4-amino-5-chloro-2-methoxybenzamido)-3-methoxypiperidin-1-yl)methyl]piperidine-1-carboxylate

455 mg of cis-4-amino-5-chloro-2-methoxy-N-(3-methoxypiperidin-4-yl)benzamide (hereinafter, referred to as “cis-norcisapride”) was dissolved in N,N-dimethylformamide (DMF) to which 435 mg of ethyl 4-(bromomethyl)piperidine-1-carboxylate, 280 mg of potassium carbonate (K₂CO₃), 48 mg of potassium iodide (KI) were then sequentially added. The reaction mixture was stirred at 80° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 222 mg (32%) of the title compound.

¹H NMR (CDCl₃): δ 8.14 (d, J=8.0 Hz, 1H), 8.02 (s, 1H), 6.25 (s, 1H), 4.48 (bs, 2H), 4.20-4.00 (m, 5H), 3.81 (s, 3H), 3.36 (bs, 4H), 2.92-2.82 (m, 1H), 2.76-2.56 (m, 3H), 2.19-2.09 (m, 4H), 1.90-1.52 (m, 5H), 1.20 (t, J=6.8 Hz, 3H), and 1.12-0.97 (m, 2H)

[α]²⁵_{D}=−0.6 (c=0.2, MeOH)

### Example 2

### Preparation of ethyl 4-[((3S,4R)-4-(4-amino-5-chloro-2-methoxybenzamido)-3-methoxypiperidin-1-yl)methyl]piperidine-1-carboxylate

Analogously to Example 1, 208 mg of the title compound was prepared from 485 mg of 4-piperidinemethanol, 0.4 mL of ethyl chloroformate and 400 mg of 4-amino-5-chloro-2-methoxy-N-((3S,4R)-3-methoxypiperidin-4-yl)benzamide (hereinafter, referred to as “(+)-norcisapride”).

[α]²⁵_{D}=+11.5 (c=0.5, MeOH)

### Example 3

### Preparation of ethyl 4-[2-(cis-4-(4-amino-5-chloro-2-methoxybenzamido)-3-methoxypiperidin-1-yl)ethyl]piperidine-1-carboxylate

Analogously to Example 1, 157 mg of the title compound was prepared from 591 mg of 4-piperidineethanol, 0.73 mL of ethylchloroformate, and 300 mg of cis-norcisapride.

¹H NMR (CDCl₃): δ 8.16 (d, J=8.4 Hz, 1H), 8.01 (s, 1H), 6.25 (s, 1H), 4.49 (bs, 2H), 4.15-3.96 (m, 4H), 3.80 (s, 3H), 3.37 (bs, 4H), 3.04-2.96 (m, 1H), 2.75-2.61 (m, 3H), 2.41-2.24 (m, 3H), 2.16-2.00 (m, 2H), 1.85-1.71 (m, 2H), 1.65-1.56 (m, 2H), 1.48-1.33 (m, 3H), 1.19 (t, J=6.8 Hz, 3H), and 1.15-1.00 (m, 2H)

### Example 4

### Preparation of ethyl 2-[2-(cis-4-(4-amino-5-chloro-2-methoxybenzamido)-3-methoxypiperidin-1-yl)ethyl]piperidine-1-carboxylate

Analogously to Example 1, 186 mg of the title compound was prepared from 1.39 g of 2-piperidineethanol, 1.37 mL of ethyl chloroformate, and 300 mg of cis-norcisapride.

¹H NMR (CDCl₃): δ 8.17 (d, J=8.0 Hz, 1H), 8.04 (s, 1H), 6.26 (s, 1H), 4.41 (bs, 2H), 4.30-4.20 (m, 1H), 4.19-4.03 (m, 3H), 4.02-3.92 (m, 1H), 3.83 (s, 3H), 3.44-3.36 (m, 3H), 3.08-2.92 (m, 1H), 2.85-2.66 (m, 2H), 2.40-2.04 (m, 4H), 2.02-1.73 (m, 4H), 1.64-1.46 (m, 6H), 1.43-1.31 (m, 1H), and 1.21 (t, J=7.2 Hz, 3H)

### Example 5

### Preparation of methyl 4-[(cis-4-(4-amino-5-chloro-2-methoxybenzamido)-3-methoxypiperidin-1-yl)methyl]piperidine-1-carboxylate

Analogously to Example 1, 187 mg of the title compound was prepared from 199 mg of 4-piperidinemethanol, 0.15 mL of methyl chloroformate, and 400 mg of cis-norcisapride.

¹H NMR (CDCl₃): δ 8.15 (d, J=8.0 Hz, 1H), 8.03 (s, 1H), 6.26 (s, 1H), 4.45 (bs, 2H), 4.16-4.05 (m, 3H), 3.82 (s, 3H), 3.64 (s, 3H), 3.37 (bs, 4H), 2.2-2.82 (m, 1H), 2.80-2.60 (m, 3H), 2.20-2.00 (m, 4H), 1.90-1.59 (m, 5H), and 1.17-0.98 (m, 2H)

### Example 6

### Preparation of propyl 4-[(cis-4-(4-amino-5-chloro-2-methoxybenzamido)-3-methoxypiperidin-1-yl)methyl]piperidine-1-carboxylate

Analogously to Example 1, 200 mg of the title compound was prepared from 250 mg of 4-piperidinemethanol, 0.27 mL of propyl chloroformate, and 500 mg of cis-norcisapride.

¹H NMR (CDCl₃): δ 8.16 (d, J=8.0 Hz, 1H), 8.06 (s, 1H), 6.26 (s, 1H), 4.38 (bs, 2H), 4.20-4.01 (m, 3H), 4.00 (t, J=6.8 Hz, 2H), 3.85 (s, 3H), 3.39 (bs, 4H), 2.95-2.84 (m, 1H), 2.80-2.60 (m, 3H), 2.22-2.09 (m, 4H), 1.87-1.57 (m, 9H), 1.14-1.01 (m, 2H), and 0.92 (t, J=7.2 Hz, 3H)

### Example 7

### Preparation of butyl 4-[(cis-4-(4-amino-5-chloro-2-methoxybenzamido)-3-methoxypiperidin-1-yl)methyl]piperidine-1-carboxylate

Analogously to Example 1, 223 mg of the title compound was prepared from 244 mg of 4-piperidinemethanol, 0.3 mL of butyl chloroformate, and 500 mg of cis-norcisapride.

¹H NMR (CDCl₃): δ 8.16 (d, J=8.0 Hz, 1H), 8.07 (s, 1H), 6.27 (s, 1H), 4.36 (bs, 2H), 4.19-3.96 (m, 5H), 3.86 (s, 3H), 3.39 (bs, 4H), 2.95-2.86 (m, 1H), 2.80-2.62 (m, 3H), 2.22-2.11 (m, 4H) 1.87-1.55 (m, 7H), 1.41-1.32 (m, 2H), 1.15-1.00 (m, 2H), and 0.91 (t, J=7.6 Hz, 3H)

### Example 8

### Preparation of isopropyl 4-[(cis-4-(4-amino-5-chloro-2-methoxybenzamido)-3-methoxypiperidin-1-yl)methyl]piperidine-1-carboxylate

Analogously to Example 1, 320 mg of the title compound was prepared from 232 mg of 4-piperidinemethanol, 2.2 mL of 1M-isopropyl chloroformate/toluene, and 400 mg of cis norcisapride.

¹H NMR (CDCl₃): δ 8.15 (d, J=7.6 Hz, 1H), 8.04 (s, 1H), 6.26 (s, 1H), 4.90-4.82 (m, 1H), 4.44 (bs, 2H), 4.20-4.00 (m, 3H), 3.83 (s, 3H), 3.37 (bs, 4H), 2.94-2.82 (m, 1H), 2.75-2.60 (m, 3H), 2.20-1.95 (m, 4H), 1.90-1.56 (m, 5H), 1.19 (d, J=5.6 Hz, 6H), and 1.12-0.99 (m, 2H)

### Example 9

### Preparation of isobutyl 4-[(cis-4-(4-amino-5-chloro-2-methoxybenzamido)-3-methoxypiperidin-1-yl)methyl]piperidine-1-carboxylate

Analogously to Example 1, 221 mg of the title compound was prepared from 371 mg of 4-piperidinemethanol, 0.46 mL of isobutyl chloroformate, and 400 mg of cis-norcisapride.

¹H NMR (CDCl₃): δ 8.16 (d, J=8.4 Hz, 1H), 8.07 (s, 1H), 6.27 (s, 1H), 4.36 (bs, 2H), 4.20-4.05 (m, 3H), 3.90-3.80 (m, 5H), 3.39 (bs, 3H), 2.95-2.84 (m, 1H), 2.76-2.63 (m, 3H), 2.22-2.11 (m, 4H), 1.94-1.65 (m, 8H), 1.15-1.00 (m, 2H), and 0.91 (d, J=6.8 Hz, 6H)

### Example 10

### Preparation of allyl 4-[(cis-4-(4-amino-5-chloro-2-methoxybenzamido)-3-methoxypiperidin-1-yl)methyl]piperidine-1-carboxylate

Analogously to Example 1, 175 mg of the title compound was prepared from 299 mg of 4-piperidinemethanol, 0.3 mL of allyl chloroformate, and 500 mg of cis-norcisapride.

¹H NMR (CDCl₃): δ 8.16 (d, J=8.0 Hz, 1H), 8.07 (s, 1H), 6.27 (s, 1H), 5.96-5.87 (m, 1H), 5.27 (dd, J=17.2 Hz, 1.2 Hz, 1H), 5.18 (dd, J=9.2 Hz, 0.9 Hz, 1H), 4.56 (dd, J=3.9 Hz, 08 Hz, 2H), 4.37 (bs, 2H), 4.25-4.08 (m, 3H), 3.88 (s, 3H), 3.40 (bs, 4H), 2.99-2.60 (m, 4H), 2.22-2.11 (m, 3H), 1.94-1.58 (m, 6H), and 1.19-1.05 (m, 2H)

### Example 11

### Preparation of 2-ethylhexyl 4-[(cis-4-(4-amino-5-chloro-2-methoxybenzamido)-3-methoxypiperidin-1-yl)methyl]piperidine-1-carboxylate

### Step 1: Preparation of 2-ethylhexyl-4-nitrophenyl carbonate

2 mL of 2-ethylhexanol was dissolved in dichloromethane, and the solution was cooled to 0° C., to which 1.89 mL of triethylamine, 1.64 g of 4-dimethylaminopyridine and 2.71 g of 4-nitrophenyl chloroformate were then sequentially added. The reaction mixture was warmed to room temperature and stirred for 12 hours, followed by addition of water and extraction with dichloromethane. The organic layer was dried over anhydrous magnesium sulfate and concentrated under reduced pressure. The residue was purified by column chromatography to afford 3.2 g (85%) of the title compound.

¹H NMR (CDCl₃): δ 8.26 (dd, J=7 Hz, 2 Hz, 2H), 7.36 (dd, J=7 Hz, 2 Hz, 2H), 4.19 (dd, J=6 Hz, 2.8 Hz, 2H), 1.68 (m, 1H), 1.45-1.26 (m, 8H), and 0.94-0.86 (m, 6H)

### Step 2: Preparation of 2-ethylhexyl-4-(hydroxymethyl)piperidine-1-carboxylate

1.13 g of 4-piperidinemethanol was dissolved in dichloromethane and the solution was cooled to 0° C., to which 3.59 mL of N,N-diisopropylethylamine (DIPEA) and 3.19 g of 2-ethylhexyl-4-nitrophenyl carbonate were then added. The reaction mixture was warmed to room temperature and stirred for 12 hours, followed by addition of water and extraction with dichloromethane. The organic layer was dried over anhydrous magnesium sulfate and concentrated under reduced pressure. The resulting residue was purified by column chromatography to afford 2.59 g (97%) of the title compound.

¹H NMR (CDCl₃): δ 4.24-4.04 (m, 2H), 3.99-3.93 (m, 2H), 3.50-3.47 (m, 2H), 2.80-2.67 (m, 2H), 1.75-1.50 (m, 4H), 1.40-1.25 (m, 8H), 1.20-1.10 (m, 2H), and 0.95-0.86 (m, 6H)

### Step 3: Preparation of 2-ethylhexyl-4-(bromomethyl)piperidine-1-carboxylate

2.59 g of 2-ethylhexyl-4-(hydroxymethyl)piperidine-1-carboxylate was dissolved in dichloromethane and the solution was cooled to 0° C., to which 2.75 g of triphenylphosphine and 1.86 g of N-bromosuccinimide were then added. The reactants were warmed to room temperature, stirred for 12 hours, and concentrated under reduced pressure. The residue was purified by column chromatography to afford 2.84 g (89%) of the title compound.

¹H NMR (CDCl₃): δ 4.22-4.02 (m, 2H), 3.98-3.93 (m, 2H), 3.26 (d, J=6.4 Hz, 2H), 2.80-2.65 (m, 2H), 1.84-1.75 (m, 3H), 1.60-1.50 (m, 1H), 1.38-1.11 (m, 10H), and 0.95-0.83 (m, 6H)

### Step 4: Preparation of 2-ethylhexyl 4-[(cis-4-(4-amino-5-chloro-2-methoxybenzamido)-3-methoxypiperidin-1-yl)methyl]piperidine-1-carboxylate

500 mg of cis-norcisapride was dissolved in N,N-dimethylformamide to which 639 mg of 2-ethylhexyl-4-(bromomethyl)piperidine-1-carboxylate, 308 mg of potassium carbonate, and 53 mg of potassium iodide were then sequentially added. The reaction mixture was stirred at 80° C. for 12 hours. After being cooled to room temperature, water was added to the reactants, followed by extraction with ethyl acetate. The extracted organic layer was dried over anhydrous magnesium sulfate and concentrated under reduced pressure. The residue was purified by column chromatography to afford 450 mg (57%) of the title compound.

¹H NMR (CDCl₃): δ 8.16 (d, J=8.0 Hz, 1H), 8.07 (s, 1H), 6.27 (s, 1H), 4.35 (bs, 2H), 4.30-4.00 (m, 3H), 3.97-3.94 (m, 2H), 3.86 (s, 3H), 3.41 (bs, 4H), 2.99-2.81 (m, 1H), 2.80-2.60 (m, 3H), 2.20-2.13 (m, 4H), 1.89-1.50 (m, 6H), 1.37-1.21 (m, 8H), 1.19-1.00 (m, 2H), and 0.92-0.85 (m, 6H)

### Example 12

### Preparation of 3-methyl-pentyl 4-[(cis-4-(4-amino-5-chloro-2-methoxybenzamido)-3-methoxypiperidin-1-yl)methyl]piperidine-1-carboxylate

Analogously to Example 11, 195 mg of the title compound was prepared from 0.56 mL of 3-methylpentanol, 957 mg of 4-nitrophenyl chloroformate, and 500 mg of cis-norcisapride.

¹H NMR (CDCl₃): δ 8.16 (d, J=8.0 Hz, 1H), 8.07 (s, 1H), 6.27 (s, 1H), 4.36 (bs, 2H), 4.24-4.01 (m, 4H), 3.85 (s, 3H), 3.39 (bs, 4H), 2.97-2.85 (m, 1H), 2.78-2.60 (m, 3H), 2.24-2.08 (m, 3H), 1.92-1.57 (m, 6H), 1.45-1.31 (m, 4H), 1.23-1.04 (m, 4H), and 0.89-0.84 (m, 6H)

### Example 13

### Preparation of 4-methyl-pentyl 4-[(cis-4-(4-amino-5-chloro-2-methoxybenzamido)-3-methoxypiperidin-1-yl)methyl]piperidine-1-carboxylate

Analogously to Example 11, 166 mg of the title compound was prepared from 0.76 mL of 4-methylpentanol, 1.29 g of 4-nitrophenyl chloroformate, and 500 mg of cis-norcisapride.

¹H NMR (CDCl₃): δ 8.15 (d, J=8.0 Hz, 1H), 8.06 (s, 1H), 6.26 (s, 1H), 4.38 (bs, 2H), 4.23-3.98 (m, 5H), 3.85 (s, 3H), 3.39 (bs, 4H), 2.94-2.84 (m, 1H), 2.78-2.58 (m, 3H), 2.21-2.10 (m, 3H), 1.87-1.50 (m, 9H), 1.23-1.17 (m, 2H), 1.11-1.03 (m, 2H), 0.86 (d, J=6.8 Hz, 6H)

### Example 14

### Preparation of cis-4-amino-5-chloro-N-[1-((1-isobutyrylpiperidin-4-yl)methyl)-3-methoxypiperidin-4-yl]-2-methoxybenzamide

### Step 1: Preparation of 1-(4-(hydroxymethyl)piperidin-1-yl)-2-methylpropan-1-one

2 mL of isobutyric acid was dissolved in dichloromethane and the solution was cooled to 0° C., to which 9.08 mL of triethylamine and 4.11 mL of ethyl chloroformate were then added. The reaction mixture was stirred for 30 min, followed by addition of 2.98 g of 4-piperidinemethanol. The reaction mixture was warmed to room temperature and stirred for 2 hours, followed by addition of water and extraction with dichloromethane. The organic layer was dried over anhydrous magnesium sulfate and concentrated under reduced pressure. The residue was purified by column chromatography to afford 1.97 g (49%) of the title compound.

¹H NMR (CDCl₃): δ 4.67-4.63 (m, 1H), 3.96-3.92 (m, 1H), 3.56-3.43 (m, 2H), 3.03-2.96 (m, 1H), 2.82-2.74 (m, 1H), 2.55-2.48 (m, 1H), 1.84-1.68 (m, 4H), and 1.23-1.07 (m, 7H)

### Step 2: Preparation of 1-(4-(bromomethyl)piperidin-1-yl)-2-methylpropan-1-one

1.97 g of 1-(4-(hydroxymethyl)piperidin-1-yl)-2-methylpropan-1-one was dissolved in dichloromethane and the solution was cooled to 0° C., to which 3.06 g of triphenylphosphine and 2.08 g of N-bromosuccinimide were then added. The reactants were warmed to room temperature, stirred for 12 hours, and concentrated under reduced pressure. The residue was purified by column chromatography to afford 1.95 g (74%) of the title compound.

¹H NMR (CDCl₃): δ 4.72-4.6 (m, 1H), 4.02-3.90 (m, 1H), 3.34-3.22 (m, 2H), 3.05-2.90 (m, 1H), 2.81-2.75 (m, 2H), 2.57-2.43 (m, 1H), 2.00-1.77 (m, 3H), and 1.28-1.00 (m, 8H)

### Step 3: Preparation of cis-4-amino-5-chloro-N-[1-((1-isobutyrylpiperidin-4-yl)methyl)-3-methoxypiperidin-4-yl]-2-methoxybenzamide

400 mg of cis-norcisapride was dissolved in N,N-dimethylformamide to which 379 mg of 1-(4-(bromomethyl)piperidin-1-yl)-2-methylpropan-1-one, 246 mg of potassium carbonate, and 42 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 195 mg (31%) of the title compound.

¹H NMR (CDCl₃): δ 8.15 (d, J=8 Hz, 1H), 8.06 (s, 1H), 6.27 (s, 1H), 4.61-4.58 (m, 1H), 4.39 (bs, 2H), 4.14-4.23 (m, 1H), 3.96-3.84 (m, 4H), 3.39 (bs, 4H), 3.03-2.84 (m, 2H), 2.80-2.74 (m, 1H), 2.70-2.60 (m, 1H), 2.57-2.44 (m, 1H), 2.22-2.15 (m, 3H), 1.92-1.66 (m, 6H), and 1.14-0.98 (m, 8H)

[α]²⁵_{D}=−0.2 (c=0.5, MeOH)

### Example 15

### Preparation of (3S,4R)-4-amino-5-chloro-N-[1-((1-isobutyrylpiperidin-4-yl)methyl)-3-methoxypiperidin-4-yl]-2-methoxybenzamide

Analogously to Example 14, 229 mg of the title compound was prepared from 0.48 mL of isobutyric acid, 717 mg of 4-piperidinemethanol, and 490 mg of (+)-norcisapride.

[α]²⁵_{D}=+12.8 (c=0.5, MeOH)

### Example 16

### Preparation of cis-4-amino-5-chloro-N-[1-(2-(1-isobutyrylpiperidin-4-yl)ethyl)-3-methoxypiperidin-4-yl]-2-methoxybenzamide

Analogously to Example 14, 118 mg of the title compound was prepared from 0.34 mL of isobutyric acid, 568 mg of 4-piperidineethanol, and 300 mg of cis-norcisapride.

¹H NMR (CDCl₃): δ 8.17 (d, J=8.4 Hz, 1H), 8.04 (s, 1H), 6.26 (s, 1H), 4.59-4.51 (m, 1H), 4.44 (bs, 2H), 4.17-4.08 (m, 1H), 3.90-2.82 (m, 4H), 339 (bs, 4H), 3.06-2.91 (m, 2H), 2.80-2.68 (m, 2H), 2.54-2.44 (m, 1H), 2.43-2.26 (m, 2H), 2.18-2.02 (m, 2H), 1.88-1.63 (m, 4H), 1.55-1.36 (m, 3H), and 1.17-1.00 (m, 8H)

### Example 17

### Preparation of cis-4-amino-5-chloro-N-[1-((1-propionylpiperidin-4-yl)methyl)-3-methoxypiperidin-4-yl]-2-methoxybenzamide

Analogously to Example 14, 115 mg of the title compound was prepared from 0.31 mL of propionic acid, 573 mg of 4-piperidinemethanol, and 300 mg of cis-norcisapride.

¹H NMR (CDCl₃): δ 8.15 (d, J=8 Hz, 1H), 8.05 (s, 1H), 6.27 (s, 1H), 4.59-4.55 (m, 1H), 4.43 (bs, 2H), 4.22-4.16 (m, 1H), 3.86-3.76 (m, 4H), 3.38 (bs, 4H), 3.00-2.85 (m, 2H), 2.70-2.60 (m, 1H), 2.55-2.48 (m, 1H), 2.30 (q, J=15 Hz, 7.2 Hz, 2H), 2.24-2.08 (m, 3H), 1.92-1.65 (m, 6H), and 1.12-0.99 (m, 5H)

### Example 18

### Preparation of cis-4-amino-5-chloro-N-[1-(2-(1-propionylpiperidin-4-yl)ethyl)-3-methoxypiperidin-4-yl]-2-methoxybenzamide

Analogously to Example 14, 83 mg of the title compound was prepared from 0.35 mL of propionic acid, 729 mg of 4-piperidineethanol, and 300 mg of cis-norcisapride.

¹H NMR (CDCl₃): δ 8.15 (d, J=8.0 Hz, 1H), 8.01 (s, 1H), 6.26 (s, 1H), 4.56-4.47 (m, 3H), 4.13-4.05 (m, 1H), 3.83-3.72 (m, 4H), 3.37 (bs, 4H), 3.04-2.88 (m, 2H), 2.76-2.66 (m, 1H), 2.52-2.43 (m, 1H), 2.40-2.25 (m, 4H), 2.16-2.00 (m, 2H), 1.85-1.61 (m, 4H), 1.53-1.36 (m, 3H), and 1.13-0.99 (m, 5H)

### Example 19

### Preparation of cis-4-amino-5-chloro-N-[1-((1-propionylpiperidin-3-yl)methyl)-3-methoxypiperidin-4-yl]-2-methoxybenzamide

Analogously to Example 14, 30 mg of the title compound was prepared from 0.28 mL of propionic acid, 651 mg of 3-piperidinemethanol, and 200 mg of cis-norcisapride.

¹H NMR (CDCl₃): δ 8.23-8.10 (m, 1H), 8.05 (s, 1H), 6.27 (s, 1H), 4.43 (bs, 2H), 4.24-4.12 (m, 2H), 3.86-3.84 (m, 3H), 3.72-3.63 (m, 1H), 3.47-3.38 (m, 4H), 3.07-2.79 (m, 2H), 2.73-2.55 (m, 1H), 2.44-2.04 (m, 5H), 1.93-1.58 (m, 5H), 1.50-1.35 (m, 2H), 1.25-1.21 (m, 2H), and 1.11 (t, J=7.6 Hz, 3H)

### Example 20

### Preparation of cis-4-amino-5-chloro-N-[1-((1-butyrylpiperidin-4-yl)methyl)-3-methoxypiperidin-4-yl]-2-methoxybenzamide

Analogously to Example 14, 215 mg of the title compound was prepared from 700 mg of sodium butyrate, 770 mg of 4-piperidinemethanol, and 500 mg of cis-norcisapride.

¹H NMR (CDCl₃): δ 8.17 (d, J=8.4 Hz, 1H), 8.07 (s, 1H), 6.27 (s, 1H), 4.64-4.56 (m, 1H), 4.36 (bs, 2H), 4.25-4.15 (m, 1H), 3.92-3.78 (m, 4H), 3.40 (bs, 4H), 3.04-2.85 (m, 2H), 2.72-2.62 (m, 1H), 2.57-2.47 (m, 1H), 2.28 (t, J=6.8 Hz, 2H), 2.22-2.09 (m, 3H), 1.92-1.58 (m, 8H), 1.10-1.02 (m, 2H), and 0.94 (t, J=7.2 Hz, 3H)

### Example 21

### Preparation of cis-4-amino-5-chloro-N-[1-((1-butyrylpiperidin-3-yl)methyl)-3-methoxypiperidin-4-yl]-2-methoxybenzamide

Analogously to Example 14, 135 mg of the title compound was prepared from 714 mg of sodium butyrate, 1.12 g of 3-piperidinemethanol, and 300 mg of cis-norcisapride.

¹H NMR (CDCl₃): δ 8.24-8.10 (m, 1H), 8.03 (s, 1H), 6.27 (s, 1H), 4.47 (bs, 2H), 4.21-4.04 (m, 2H), 3.92-3.65 (m, 4H), 3.52-3.32 (m, 4H), 3.06-2.79 (m, 2H), 2.73-2.52 (m, 1H), 2.40-2.04 (m, 5H), 1.96-1.55 (m, 5H), 1.48-1.34 (m, 2H), 1.30-1.08 (m, 4H), and 1.04-0.88 (m, 3H)

### Example 22

### Preparation of cis-4-amino-5-chloro-N-[1-((1-pentanoylpiperidin-4-yl)methyl)-3-methoxypiperidin-4-yl]-2-methoxybenzamide

Analogously to Example 14, 296 mg of the title compound was prepared from 0.36 mL of valeric acid, 403 mg of 4-piperidinemethanol, and 500 mg of cis-norcisapride.

¹H NMR (CDCl₃): δ 8.16 (d, J=8.0 Hz, 1H), 8.07 (s, 1H), 6.27 (s, 1H), 4.60-4.56 (m, 1H), 4.38 (bs, 2H), 4.22-4.12 (m, 1H), 3.85-3.77 (m, 4H), 3.39 (bs, 4H), 3.01-2.84 (m, 2H), 2.72-2.60 (m, 1H), 2.58-2.46 (m, 1H), 2.29 (t, J=7.6 Hz, 2H), 2.24-2.08 (m, 3H), 1.92-1.64 (m, 6H), 1.61-1.54 (m, 2H), 1.36-1.31 (m, 2H), 1.15-0.98 (m, 2H), and 0.90 (t, J=7.2 Hz, 3H)

### Example 23

### Preparation of cis-4-amino-5-chloro-N-[1-((1-hexanoylpiperidin-4-yl)methyl)-3-methoxypiperidin-4-yl]-2-methoxybenzamide

Analogously to Example 14, 242 mg of the title compound was prepared from 0.41 mL of hexanoic acid, 396 mg of 4-piperidinemethanol, and 500 mg of cis-norcisapride.

¹H NMR (CDCl₃): δ 8.17d, J=8.4 Hz, 1H), 8.07 (s, 1H), 6.27 (s, 1H), 4.60-4.56 (m, 1H), 4.37 (bs, 2H), 4.24-4.13 (m, 1H), 3.90-3.76 (m, 4H), 3.40 (bs, 4H), 3.02-2.84 (m, 2H), 2.71-2.62 (m, 1H), 2.58-2.44 (m, 1H) 2.28 (t, J=8.0 Hz, 2H), 2.24-2.05 (m, 3H), 1.92-1.55 (m, 8H), 1.39-1.10 (m, 4H), 1.14-0.97 (m, 2H), and 0.87 (t, J=6.8 Hz, 3H)

### Example 24

### Preparation of cis-4-amino-5-chloro-N-[1-((2-methylpentanoyl)piperidin-4-yl)methyl)-3-methoxypiperidin-4-yl]-2-methoxybenzamide

Analogously to Example 14, 580 mg of the title compound was prepared from 0.8 mL of 2-methylvaleric acid, 776 mg of 4-piperidinemethanol, and 800 mg of cis-norcisapride.

¹H NMR (CDCl₃): δ 8.17 (d, J=8.0 Hz, 1H), 8.07 (s, 1H), 6.27 (s, 1H), 4.68-4.58 (m, 1H), 4.36 (bs, 2H), 4.23-4.13 (m, 1H), 3.96-3.90 (m, 1H), 3.86 (s, 3H), 3.40 (bs, 4H), 3.04-2.86 (m, 2H), 2.74-2.63 (m, 2H), 2.57-2.47 (m, 1H), 2.26-2.09 (m, 4H), 1.92-1.56 (m, 6H), 1.38-1.20 (m, 3H), 1.16-0.99 (m, 5H), and 0.90-0.85 (m, 3H)

### Example 25

### Preparation of cis-4-amino-5-chloro-N-[1-((1-(3-methylbutanoyl)piperidin-4-yl)methyl)-3-methoxypiperidin-4-yl]-2-methoxybenzamide

Analogously to Example 14, 148 mg of the title compound was prepared from 0.33 mL of isovaleric acid, 371 mg of 4-piperidinemethanol, and 500 mg of cis-norcisapride.

¹H NMR (CDCl₃): δ 8.17 (d, J=8.0 Hz, 1H), 8.07 (s, 1H), 6.27 (s, 1H), 4.64-4.57 (m, 1H), 4.37 (bs, 2H), 4.24-4.16 (m, 1H), 3.93-3.78 (m, 4H), 3.40 (bs, 4H), 3.02-2.84 (m, 2H), 2.72-2.60 (m, 1H), 2.56-2.47 (m, 1H), 2.25-2.00 (m, 6H), 1.92-1.65 (m, 6H), 1.14-1.00 (m, 2H), and 0.94 (d, J=6.8 Hz, 6H)

### Example 26

### Preparation of cis-4-amino-5-chloro-N-[1-((1-(3,3-dimethylbutanoyl)piperidin-4-yl)methyl)-3-methoxypiperidin-4-yl]-2-methoxybenzamide

Analogously to Example 14, 202 mg of the title compound was prepared from 0.58 mL of 3,3-dimethylbutyric acid, 546 mg of 4-piperidinemethanol, and 500 mg of cis-norcisapride.

¹H NMR (CDCl₃): δ 8.17 (d, J=8.0 Hz, 1H), 8.07 (s, 1H), 6.27 (s, 1H), 4.68-4.61 (m, 1H), 4.36 (bs, 2H), 4.23-4.13 (m, 1H), 3.97-3.82 (m, 4H), 3.40 (bs, 4H), 3.03-2.85 (m, 2H), 2.72-2.62 (m, 1H), 2.56-2.44 (m, 1H), 2.27-2.10 (m, 5H), 1.92-1.61 (m, 6H), and 1.15-0.96 (m, 11H)

### Example 27

### Preparation of cis-4-amino-5-chloro-N-[1-((1-(4-methylpentanoyl)piperidin-4-yl)methyl)-3-methoxypiperidin-4-yl]-2-methoxybenzamide

Analogously to Example 14, 252 mg of the title compound was prepared from 0.36 mL of 4-methylvaleric acid, 345 mg of 4-piperidinemethanol, and 500 mg of cis-norcisapride.

¹H NMR (CDCl₃): δ 8.15 (d, J=7.6 Hz, 1H), 8.06 (s, 1H), 6.27 (s, 1H), 4.59-4.55 (m, 2H), 4.40 (bs, 2H), 4.24-4.12 (m, 1H), 3.91-3.73 (m, 4H), 3.39 (bs, 4H), 3.05-2.81 (m, 2H), 2.72-2.45 (m, 1H), 2.29 (t, J=7.6 Hz, 2H), 2.23-2.06 (m, 3H), 1.95-1.65 (m, 6H), 1.61-1.42 (m, 3H), 1.15-0.98 (m, 2H), and 0.88 (d, J=6.0 Hz, 6H)

### Example 28

### Preparation of cis-4-amino-5-chloro-N-[1-((1-acetylpiperidin-4-yl)methyl)-3-methoxypiperidin-4-yl]-2-methoxybenzamide

### Step 1: Preparation of 1-(4-(hydroxymethyl)piperidin-1-yl)ethanone

1 mL of acetic acid was dissolved in dichloromethane and the solution was cooled to 0° C., to which 7.3 mL of triethylamine and 3.3 mL, of ethyl chloroformate were then added. The reaction mixture was stirred for 1 hour, followed by addition of 2.19 g of 4-piperidinemethanol. The reaction mixture was warmed to room temperature and stirred for 5 hours, followed by addition of water and extraction with dichloromethane. The organic layer was dried over anhydrous magnesium sulfate and concentrated under reduced pressure. The residue was purified by column chromatography to afford 1.04 g (38%) of the title compound.

¹H NMR (CDCl₃): δ 4.62 (ddd, J=13.2 Hz, 2.4 Hz, 2 Hz, 1H), 3.82 (ddd, J=13.6 Hz, 2.4 Hz, 1.6 Hz, 1H), 3.56-3.44 (m, 2H), 3.03 (ddd, J=13.0 Hz, 13.0 Hz, 2.4 Hz, 1H), 2.53 (ddd, J=13.0 Hz, 13.0 Hz, 2.8 Hz, 1H), 2.07 (s, 3H), 1.85-1.70 (m, 3H), and 1.24-1.06 (m, 2H)

### Step 2: Preparation of (1-acetylpiperidin-4-yl)methyl methanesulfonate

1.04 g of 1-(4-(hydroxymethyl)piperidin-1-yl)ethanone was dissolved in dichloromethane and the solution was cooled to 0° C., to which 1.39 mL of triethylamine and 0.76 mL of methanesulfonyl chloride were then added. The reactants were warmed to room temperature and stirred for 6 hours, followed by addition of water and extraction with dichloromethane. The organic layer was dried over anhydrous magnesium sulfate and concentrated under reduced pressure. The residue was purified by column chromatography to afford 1.02 g (62%) of the title compound.

¹H NMR (CDCl₃): δ 4.68-4.07 (m, 1H), 4.07-4.05 (m, 2H), 3.86-3.82 (m, 1H), 3.08-2.99 (m, 4H), 2.58-2.48 (m, 1H), 2.07 (s, 3H), 2.05-1.93 (m, 1H), 1.86-1.74 (m, 2H), and 1.28-1.14 (m, 2H)

### Step 3: Preparation of cis-4-amino-5-chloro-N-[1-((1-acetylpiperidin-4-yl)methyl)-3-methoxypiperidin-4-yl]-2-methoxybenzamide

400 mg of cis-norcisapride was dissolved in N,N-dimethylformamide to which 360 mg of (1-acetylpiperidin-4-yl)methyl methanesulfonate, 246 mg of potassium carbonate, and 42 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 27 mg (5%) of the title compound.

¹H NMR (CDCl₃): δ 8.15 (d, J=8.0 Hz, 1H), 8.05 (s, 1H), 6.26 (s, 1H), 4.60-4.50 (m, 1H), 4.42 (bs, 2H), 4.21-4.11 (m, 1H), 3.84 (s, 3H), 3.80-3.72 (m, 1H), 3.38 (bs, 4H), 3.08-2.84 (m, 2H), 2.70-2.60 (m, 1H), 2.57-2.46 (m, 1H), 2.25-1.61 (m, 12H), and 1.15-0.96 (m, 2H)

### Example 29

### Preparation of cis-4-amino-5-chloro-N-[1-((1-pivaloylpiperidin-4-yl)methyl)-3-methoxypiperidin-4-yl]-2-methoxybenzamide

### Step 1: Preparation of 1-(4-(hydroxymethyl)piperidin-1-yl)-2,2-dimethylpropan-1-one

1 g of 4-piperidinemethanol was dissolved in dichloromethane and the solution was cooled to 0° C., to which 3.18 mL of N,N-diisopropylethylamine and 1.12 mL of pivaloyl chloride were then added. The reaction mixture was warmed to room temperature and stirred for 12 hours, followed by addition of water and extraction with dichloromethane. The organic layer was dried over anhydrous magnesium sulfate and concentrated under reduced pressure. The residue was purified by column chromatography to afford 1.48 g (86%) of the title compound.

¹H NMR (CDCl₃): δ 4.45-4.41 (m, 2H), 3.49 (d, J=12.4 Hz, 2H), 2.81-2.72 (m, 2H), 1.81-1.70 (m, 3H), 1.26 (s, 9H), and 1.22-1.13 (m, 2H)

### Step 2: Preparation of 1-(4-bromomethyl)piperidin-1-yl)-2,2-dimethylpropan-1-one

1.48 g of 1-(4-(hydroxymethyl)piperidin-1-yl)-2,2-dimethylpropan-1-one was dissolved in dichloromethane and the solution was cooled to 0° C., to which 2.14 g of triphenylphosphine and 1.46 g of N-bromosuccinimide were then added. The reactants were warmed to room temperature, stirred for 12 hours, and concentrated under reduced pressure. The residue was purified by column chromatography to afford 1.61 g (83%) of the title compound.

¹H NMR (CDCl₃): δ 4.42-4.38 (m, 2H), 3.24 (dd, J=6.0 Hz, 2.4 Hz, 2H), 2.78-2.66 (m, 2H), 1.92-1.79 (m, 3H), and 1.28-1.06 (m, 11H)

### Step 3: Preparation of cis-4-amino-5-chloro-N-[1-((1-pivaloylpiperidin-4-yl)methyl)-3-methoxypiperidin-4-yl]-2-methoxybenzamide

500 mg of cis-norcisapride was dissolved in N,N-dimethylformamide to which 501 mg of 1-(4-(bromomethyl)piperidin-1-yl)-2,2-dimethylpropan-1-one, 308 mg of potassium carbonate, and 53 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 228 mg (29%) of the title compound.

¹H NMR (CDCl₃): δ 8.16 (d, J=8.0 Hz, 1H), 8.05 (s, 1H), 6.27 (s, 1H), 4.49-4.31 (m, 4H), 4.24-4.13 (m, 1H), 3.84 (s, 3H), 3.39 (bs, 4H), 2.98-2.84 (m, 1H), 2.82-2.60 (m, 3H), 2.21-2.08 (m, 3H), 1.95-1.68 (m, 6H), 1.24 (s, 9H), and 1.16-1.00 (m, 2H)

### Example 30

### Preparation of cis-4-amino-5-chloro-N-[1-((1-(2-methylpropanethioyl)piperidin-4-yl)methyl)-3-methoxypiperidin-4-yl]-2-methoxybenzamide

### Step 1: Preparation of 1-(4-(bromomethyl)piperidin-1-yl)-2-methylpropan-1-thione

740 mg of 1-(4-(bromomethyl)piperidin-1-yl)-2-methylpropan-1-one was dissolved in tetrahydrofuran (THF), and the solution was cooled to 0° C., followed by addition of 724 mg of Lawesson's reagent. The reactants were warmed to room temperature and stirred for 30 min, followed by stirring under reflux for another 20 hours. After being cooled to room temperature, extraction was carried out with ethyl acetate. The organic layer was dried over anhydrous magnesium sulfate and concentrated under reduced pressure. The resulting residue was purified by column chromatography to afford 730 mg (93%) of the title compound.

¹H NMR (CDCl₃): δ 5.79-5.74 (m, 1H), 4.48-4.00 (m, 1H), 3.35-3.09 (m, 4H), 2.92 (td, J=12.8 Hz, 2.8 Hz, 1H), 2.10-1.98 (m, 2H), 1.96-1.88 (m, 1H), and 1.46-1.12 (m, 7H)

### Step 2: Preparation of cis-4-amino-5-chloro-N-[1-((1-(2-methylpropanethioyl)piperidin-4-yl)methyl)-3-methoxypiperidin-4-yl]-2-methoxybenzamide

722 mg of cis-norcisapride was dissolved in N,N-dimethylformamide to which 730 mg of 1-(4-(bromomethyl)piperidin-1-yl)-2-methylpropan-1-thione, 480 mg of potassium carbonate, and 74 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 380 mg (33%) of the title compound.

¹H NMR (CDCl₃): δ 8.15 (d, J=8.0 Hz, 1H), 8.08 (s, 1H), 6.27 (s, 1H), 5.69-5.64 (m, 1H), 4.44-4.30 (m, 3H), 4.24-4.15 (m, 1H), 3.87 (s, 3H), 3.40 (bs, 4H), 3.22-3.07 (m, 2H), 3.03-2.84 (m, 2H), 2.75-2.63 (m, 1H), 2.28-2.10 (m, 4H), 2.01-1.71 (m, 5H), and 1.35-1.08 (m, 8H)

### Example 31

### Preparation of cis-4-amino-5-chloro-N-[1-((1-ethanethioylpiperidin-4-yl)methyl)-3-methoxypiperidin-4-yl]-2-methoxybenzamide

### Step 1: Preparation of (1-ethanethioylpiperidin-4-yl)methyl methanesulfonate

660 mg of (1-acetylpiperidin-4-yl)methyl methanesulfonate was dissolved in tetrahydrofuran and the solution was cooled to 0° C., followed by addition of 681 mg of Lawesson's reagent. The reactants were warmed to room temperature and stirred for 30 min, followed by stirring under reflux for another 20 hours. After being cooled to room temperature, extraction was carried out with ethyl acetate. The organic layer was dried over anhydrous magnesium sulfate and concentrated under reduced pressure. The resulting residue was purified by column chromatography to afford 590 mg (84%) of the title compound.

¹H NMR (CDCl₃): δ 5.67-5.63 (m, 1H), 4.27-4.23 (m, 1H), 4.12-4.03 (m, 2H), 3.2 (td, J=13.2 Hz, 3.2 Hz, 1H), 3.02-2.91 (m, 4H), 2.64 (s, 3H), 2.19-2.06 (m, 1H), 1.94-1.84 (m, 2H), and 1.48-1.28 (m, 2H)

### Step 2: Preparation of cis-4-amino-5-chloro-N-[1-((1-ethanethioylpiperidin-4-yl)methyl)-3-methoxypiperidin-4-yl]-2-methoxybenzamide

614 mg of cis-norcisapride was dissolved in N,N-dimethylformamide to which 590 mg of (1-ethanethioylpiperidin-4-yl)methyl methanesulfonate, 405 mg of potassium carbonate, and 65 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 290 mg (32%) of the title compound.

¹H NMR (CDCl₃): δ 8.16 (d, J=8.0 Hz, 1H), 8.07 (s, 1H), 6.27 (s, 1H), 5.58-5.51 (m, 1H), 4.36 (bs, 2H), 4.23-4.13 (m, 2H), 3.86 (s, 3H), 3.43 (m, 4H), 3.22-3.13 (m, 1H), 3.03-2.85 (m, 2H), 2.73-2.62 (m, 4H), 2.24-2.12 (m, 4H), 1.98-1.72 (m, 5H), and 1.36-1.12 (m, 2H)

### Example 32

### Preparation of cis-4-amino-5-chloro-N-[1-((1-propanethioylpiperidin-4-yl)methyl)-3-methoxypiperidin-4-yl]-2-methoxybenzamide

Analogously to Example 30, 400 mg of the title compound was prepared from 760 mg of 1-(4-(bromomethyl)piperidin-1-yl)propan-1-one, 788 mg of Lawesson's reagent, and 770 mg of cis-norcisapride.

¹H NMR (CDCl₃): δ 8.15 (d, J=8.0 Hz, 1H), 8.05 (s, 1H), 6.27 (s, 1H), 5.59-5.52 (m, 1H), 4.39 (bs, 2H), 4.22-4.14 (m, 2H), 3.85 (s, 3H), 3.39 (m, 4H), 3.19-3.12 (m, 1H), 2.99-2.82 (m, 4H), 2.72-2.61 (m, 1H), 2.25-2.12 (m, 4H), 1.98-1.76 (m, 5H), and 1.30-1.15 (m, 5H)

### Example 33

### Preparation of cis-4-amino-5-chloro-N-[1-((1-pentanethioylpiperidin-4-yl)methyl)-3-methoxypiperidin-4-yl]-2-methoxybenzamide

Analogously to Example 30, 510 mg of the title compound was prepared from 1.11 g of 1-(4-(bromomethyl)piperidin-1-yl)pentan-1-one, 1.71 g of Lawesson's reagent, and 921 mg of cis-norcisapride.

¹H NMR (CDCl₃): δ 8.15 (d, J=8.0 Hz, 1H), 8.06 (s, 1H), 6.27 (s, 1H), 5.57-5.52 (m, 1H), 4.38 (bs, 2H), 4.22-4.13 (m, 2H), 3.85 (s, 3H), 3.39 (bs, 4H), 3.20-3.09 (m, 1H), 2.98-2.80 (m, 4H), 2.72-2.60 (m, 1H), 2.25-2.09 (m, 4H), 2.00-1.57 (m, 7H), 1.38 (q, J=7.2 Hz, 2H), 1.32-1.11 (m, 2H), and 0.91 (t, J=7.2 Hz, 3H)

### Example 34

### Preparation of cis-4-amino-5-chloro-N-[1-((1-hexanethioylpiperidin-4-yl)methyl)-3-methoxypiperidin-4-yl]-2-methoxybenzamide

Analogously to Example 30, 390 mg of the title compound was prepared from 660 mg of 1-(4-(bromornethyl)piperidin-1-yl)hexan-1-one, 967 mg of Lawesson's reagent, and 590 mg of cis-norcisapride.

¹H NMR (CDCl₃): δ 8.15 (d, J=8.0 Hz, 1H), 8.06 (s, 1H), 6.27 (s, 1H), 5.59-5.51 (m, 1H), 4.37 (bs, 2H), 4.22-4.08 (m, 2H), 3.84 (s, 3H), 3.39 (bs, 4H), 3.20-3.10 (m, 1H), 2.99-2.76 (m, 4H), 2.72-2.61 (m, 1H), 2.25-2.08 (m, 4H), 1.97-1.56 (m, 7H), 1.40-1.12 (m, 6H), and 0.88 (t, J=7.2 Hz, 3H)

### Example 35

### Preparation of cis-4-amino-5-chloro-N-[1-((1-butanethioylpiperidin-4-yl)methyl)-3-methoxypiperidin-4-yl]-2-methoxybenzamide

Analogously to Example 30, 230 mg of the title compound was prepared from 350 mg of 1-(4-(bromomethyl)piperidin-1-yl)butan-1-one, 575 mg of Lawesson's reagent, and 331 mg of cis-norcisapride.

¹H NMR (CDCl₃): δ 8.15 (d, J=8.0 Hz, 1H), 8.07 (s, 1H), 6.27 (s, 1H), 5.60-5.52 (m, 1H), 4.36 (bs, 2H), 4.24-4.12 (m, 2H), 3.86 (s, 3H), 3.41 (bs, 4H), 3.21-3.09 (m, 1H), 3.00-2.76 (m, 4H), 2.73-2.62 (m, 1H), 2.28-2.09 (m, 4H), 2.01-1.57 (m, 7H), 1.38-1.13 (m, 2H), and 0.99 (1, J=7.2 Hz, 3H)

### Example 36

### Preparation of cis-4-amino-5-chloro-N-[1 ((1-(3-methylbutanethioyl)piperidin-4-yl)methyl)-3-methoxypiperidin-4-yl]-2-methoxybenzamide

Analogously to Example 30, 600 mg of the title compound was prepared from 1.21 g of 1-(4-(bromomethyl)piperidin-1-yl)-3-methylbutan-1-one, 1.87 g of Lawesson's reagent, and 930 mg of cis-norcisapride.

¹H NMR (CDCl₃): δ 8.15 (d, J=8.4 Hz, 1H), 8.06 (s, 1H), 6.27 (s, 1H), 5.65-5.57 (m, 1H), 4.38 (bs, 2H), 4.26-4.13 (m, 2H), 3.85 (s, 3H), 3.39 (bs, 4H), 3.21-3.12 (m, 1H), 3.02-2.83 (m, 2H), 2.76 (d, J=7.2 Hz, 2H), 2.71-2.63 (m, 1H), 2.25-2.06 (m, 5H), 1.98-1.71 (m, 5H), 1.33-1.109m, 2H), and 0.98 (d, J=6.4 Hz, 6H)

### Example 37

### Preparation of cis-4-amino-5-chloro-N-[1-((1-(4-methylpentanethioyl)piperidin-4-yl)methyl)-3-methoxypiperidin-4-yl]-2-methoxybenzamide

Analogously to Example 30, 400 mg of the title compound was prepared from 740 mg of 1-(4-(bromomethyl)piperidin-1-yl)-4-methylpentan-1-one, 1.2 g of Lawesson's reagent, and 582 mg of cis-norcisapride.

¹H NMR (CDCl₃): δ 8.15 (d, J=8.0 Hz, 1H), 8.06 (s, 1H), 6.27 (s, 1H), 5.58-5.54 (m, 1H), 4.38 (bs, 2H), 4.24-4.12 (m, 2H), 3.86 (s, 3H), 3.40 (bs, 4H), 3.21-3.11 (m, 1H), 3.00-2.91 (m, 2H), 2.86 (t, J=8.8 Hz, 2H), 2.77-2.64 (m, 1H), 2.35-2.08 (m, 4H), 2.04-1.70 (m, 5H), 1.68-1.46 (m, 3H), 1.36-1.13 (m, 2H), and 0.91 (d, J=6.4 Hz, 6H)

### Example 38

### Preparation of cis-4-amino-5-chloro-N-[1 ((1-(2,2-dimethylpropanethioyl)piperidin-4-yl)methyl)-3-methoxypiperidin-4-yl]-2-methoxybenzamide

Analogously to Example 30, 657 mg of the title compound was prepared from 938 mg of 1-(4-(bromomethyl)piperidin-1-yl)-2,2-dimethylpropan-1-one, 1.6 g of Lawesson's reagent, and 620 mg of cis-norcisapride.

¹H NMR (CDCl₃): δ 8.15 (d, J=7.6 Hz, 1H), 8.06 (s, 1H), 6.27 (s, 1H), 5.28-4.96 (m, 2H), 4.38 (bs, 2H), 4.25-4.12 (m, 1H), 3.86 (s, 3H), 3.40 (bs, 4H), 3.19-3.04 (m, 2H), 3.00-2.85 (m, 1H), 2.80-2.60 (m, 1H), 2.41-2.04 (m, 4H), 2.02-1.65 (m, 5H), and 1.52-1.10 (m, 11H)

### Example 39

### Preparation of S-ethyl 4-[(cis-4-(4-amino-5-chloro-2-methoxybenzamido)-3-methoxypiperidin-1-yl)methyl]piperidine-1-carbothioate

### Step 1: Preparation of S-ethyl O-4-nitrophenyl carbonothioate

1.19 mL of ethanethiol was dissolved in dichloromethane, and the solution was cooled to 0° C., to which 2.4 mL of triethylamine, 2.1 g of 4-dimethylaminopyridine (DMAP), and 3.41 g of 4-nitrophenyl chloroformate were then sequentially added. The reaction mixture was warmed to room temperature and stirred for 12 hours, followed by addition of water and extraction with dichloromethane. The organic layer was dried over anhydrous magnesium sulfate and concentrated under reduced pressure. The residue was purified by column chromatography to afford 1.7 g (47%) of the title compound.

¹H NMR (CDCl₃): δ 8.25 (d, J=9.2 Hz, 2H), 7.33 (d, J=9.2 Hz, 2H), 2.96 (q, J=7.2 Hz, 2H), and 1.38 (t, J=7.2 Hz, 3H)

### Step 2: Preparation of S-ethyl 4-(hydroxymethyl)piperidine-1-carbothioate

760 mg of 4-piperidinemethanol was dissolved in dichloromethane and the solution was cooled to 0° C., to which 2.42 mL of N,N-diisopropylethylamine and 1.65 g of S-ethyl O-4-nitrophenyl carbonothioate were then added. The reaction mixture was warmed to room temperature and stirred for 12 hours, followed by addition of water and extraction with dichloromethane. The organic layer was dried over anhydrous magnesium sulfate and concentrated under reduced pressure. The resulting residue was purified by column chromatography to afford 1.17 g (87%) of the title compound.

¹H NMR (CDCl₃): δ 4.60-4.34 (m, 1H), 3.98-3.74 (m, 1H), 3.44 (bs, 2H), 2.96-2.56 (m, 4H), 2.10 (bs, 1H), 1.79-1.43 (m, 3H), 1.23 (t, J=7.2 Hz, 3H), and 1.18-1.12 (m, 2H)

### Step 3: Preparation of S-ethyl 4-(bromomethyl)piperidine-1-carbothioate

1.17 g of S-ethyl 4-(hydroxymethyl)piperidine-1-carbothioate was dissolved in dichloromethane and the solution was cooled to 0° C., to which 1.66 g of triphenylphosphine and 1.13 g of N-bromosuccinimide were then added. The reactants were warmed to room temperature, stirred for 12 hours, and concentrated under reduced pressure. The residue was purified by column chromatography to afford 1.35 g (88%) of the title compound.

¹H NMR (CDCl₃): δ 4.62-4.32 (m, 1H), 4.12-3.78 (m, 1H), 3.25 (d, J=5.6 Hz, 2H), 2.96-2.52 (m, 4H), 1.90-1.76 (m, 3H), and 1.32-1.16 (m, 5H)

### Step 4: Preparation of S-ethyl 4-[(cis-4-(4-amino-5-chloro-2-methoxybenzamido)-3-methoxypiperidin-1-yl)methyl]piperidine-1-carbothioate

1.2 g of cis-norcisapride was dissolved in N,N-dimethylformamide to which 1.22 g of S-ethyl 4-(bromomethyl)piperidine-1-carbothioate, 792 mg of potassium carbonate, and 127 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 700 mg (37%) of the title compound.

¹H NMR (CDCl₃): δ 8.16 (d, J=8.0 Hz, 1H), 8.06 (s, 1H), 6.27 (s, 1H), 4.38 (bs, 2H), 4.22-4.13 (m, 1H), 3.85 (s, 3H), 3.39 (bs, 4H), 2.97-2.56 (m, 7H), 2.24-2.11 (m, 4H), 1.91-1.65 (m, 6H), 1.26 (t, J=7.6 Hz, 3H), and 1.18-1.04 (m, 2H)

### Example 40

### Preparation of S-propyl 4-[(cis-4-(4-amino-5-chloro-2-methoxybenzamido)-3-methoxypiperidin-1-yl)methyl]piperidine-1-carbothioate

Analogously to Example 39, 600 mg of the title compound was prepared from 1 mL of propane-1-thiol, 2.34 g of 4-nitrophenyl chloroformate, and 1.12 g of cis-norcisapride.

¹H NMR (CDCl₃): δ 8.15 (d, J=8.0 Hz, 1H), 8.05 (s, 1H), 6.26 (s, 1H), 4.40 (bs, 2H), 4.22-4.12 (m, 1H), 3.84 (s, 3H), 3.38 (bs, 4H), 2.96-2.56 (m, 7H), 2.23-2.08 (m, 4H), 1.90-1.66 (m, 6H), 1.63-1.58 (m, 2H), 1.17-1.03 (m, 2H), and 0.95 (t, J=6.8 Hz, 3H)

### Example 41

### Preparation of S-butyl 4-[(cis-4-(4-amino-5-chloro-2-methoxybenzamido)-3-methoxypiperidin-1-yl)methyl]piperidine-1-carbothioate

Analogously to Example 39, 1 g of the title compound was prepared from 1 mL of butane-1-thiol, 1.97 g of 4-nitrophenyl chloroformate, and 1.1 g of cis-norcisapride.

¹H NMR (CDCl₃): δ 8.15 (d, J=8.0 Hz, 1H), 8.05 (s, 1H), 6.26 (s, 1H), 4.40 (bs, 2H), 4.20-4.11 (m, 1H), 3.84 (s, 3H), 3.38 (bs, 4H), 2.98-2.53 (m, 7H), 2.23-2.08 (m, 4H), 1.91-1.62 (m, 6H), 1.58-1.52 (m, 2H), 1.40-1.34 (m, 2H), 1.17-1.02 (m, 2H), and 0.88 (t, J=7.2 Hz, 3H)

### Example 42

### Preparation of cis-4-amino-5-chloro-N-[1-((1-(isopropylsulfonyl)piperidin-4-yl)methyl)-3-methoxypiperidin-4-yl]-2-methoxybenzamide

### Step 1: Preparation of (1-(isopropylsulfonyl)piperidin-4-yl)methyl isopropylsulfonate

1 g of 4-piperidinemethanol was dissolved in dichloromethane, and the solution was cooled to 0° C. Then, 2.56 mL of triethylamine was added followed by slow addition of 2.05 mL of 2-propanesulfonyl chloride. The reaction mixture was warmed to room temperature and stirred for 2 hours, followed by addition of water and extraction with dichloromethane. The organic layer was dried over anhydrous magnesium sulfate, concentrated under reduced pressure, and purified by column chromatography to afford 1.99 g (70%) of the title compound.

¹H NMR (CDCl₃): δ 4.05 (d, J=6.8 Hz, 2H), 3.88-3.80 (m, 2H), 3.31-3.22 (m, 1H, 3.18-3.11 (m, 1H), 2.89-2.82 (m, 2H), 1.96-1.76 (m, 3H), 1.40 (d, J=6.8 Hz, 6H), and 1.36-1.29 (m, 8H)

### Step 2: Preparation of cis-4-amino-5-chloro-N-[1-((1-(isopropylsulfonyl)piperidin-4-yl)methyl)-3-methoxypiperidin-4-yl]-2-methoxybenzamide

400 mg of cis-norcisapride was dissolved in N,N-dimethylformamide to which 501 mg of (1-(isopropylsulfonyl)piperidin-4-yl)methyl isopropyl sulfonate, 246 mg of potassium carbonate, and 42 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 174 mg (26%) of the title compound.

¹H NMR (CDCl₃): δ 8.15 (d, J=8.4 Hz, 1H), 8.02 (s, 1H), 6.27 (s, 1H), 4.47 (bs, 2H), 4.17-4.09 (m, 1H), 3.82 (s, 3H), 3.78-3.70 (m, 2H), 3.36 (bs, 4H), 3.15-3.08 (m, 1H), 2.91-2.76 (m, 3H), 2.67-2.57 (m, 1H), 2.21-2.10 (m, 4H), 1.88-1.69 (m, 4H), 1.64-1.53 (m, 1H), 1.28 (d, J=6.8 Hz, 6H), and 1.25-1.13 (m, 2H)

### Example 43

### Preparation of cis-4-amino-5-chloro-N-[1 ((1-(methylsulfonyl)piperidin-4-yl)methyl)-3-methoxypiperidin-4-yl]-2-methoxybenzamide

Analogously to Example 42, 40 mg of the title compound was prepared from 435 mg of 4-piperidinemethanol, 0.73 mL of methanesulfonyl chloride, and 300 mg of cis-norcisapride.

¹H NMR (CDCl₃): δ 8.15 (d, J=8.0 Hz, 1H), 8.04 (s, 1H), 6.27 (s, 1H), 4.41 (bs, 2H), 4.20-4.10 (m, 1H), 3.84 (s, 3H), 3.78-3.70 (m, 2H), 3.41 (bs, 4H), 2.94-2.82 (m, 1H), 2.73 (s, 3H), 2.68-2.56 (m, 3H), 2.24-2.10 (m, 3H), 1.95-1.65 (m, 5H), 1.64-1.51 (m, 1H), and 1.30-1.12 (m, 2H)

### Example 44

### Preparation of (3S,4R)-4-amino-5-chloro-N-[1-((1-(methylsulfonyl)piperidin-4-yl)methyl)-3-methoxypiperidin-4-yl]-2-methoxybenzamide

Analogously to Example 42, 186 mg of the title compound was prepared from 762 mg of 4-piperidinemethanol, 1.28 mL of methanesulfonyl chloride, and 525 mg of (+)-norcisapride.

¹H NMR (CDCl₃): δ 8.15 (d, J=8.0 Hz, 1H), 8.04 (s, 1H), 6.27 (s, 1H), 4.41 (bs, 2H), 4.20-4.10 (m, 1H), 3.84 (s, 3H), 3.78-3.70 (m, 2H), 3.41 (bs, 4H), 2.94-2.82 (m, 1H), 2.73 (s, 3H), 2.68-2.56 (m, 3H), 2.24-2.10 (m, 3H), 1.95-1.65 (m, 5H), 1.64-1.51 (m, 1H), and 1.30-1.12 (m, 2H)

### Example 45

### Preparation of cis-4-amino-5-chloro-N-[1-(2-(1-(methylsulfonyl)piperidin-4-yl)ethyl)-3-methoxypiperidin-4-yl]-2-methoxybenzamide

Analogously to Example 42, 281 mg of the title compound was prepared from 386 mg of 4-piperidineethanol, 0.58 mL of methanesulfonyl chloride, and 400 mg of cis-norcisapride.

¹H NMR (CDCl₃): δ 8.16 (d, J=8.0 Hz, 1H), 8.03 (s, 1H), 6.26 (s, 1H), 4.43 (bs, 2H), 4.17-4.08 (m, 1H), 3.83 (s, 3H), 4.76-4.67 (m, 2H), 3.39 (bs, 4H), 3.05-2.92 (m, 1H), 2.72 (bs, 4H), 2.63-2.56 (m, 2H), 2.42-2.28 (m, 2H), 2.18-2.03 (m, 2H), 1.88-1.72 (m, 4H), and 1.55-1.20 (m, 5H)

### Example 46

### Preparation of cis-4-amino-5-chloro-N-[1-(3-(1H-1,2,4-triazol-1-yl)propyl)-3-methoxypiperidin-4-yl]-2-methoxybenzamide

### Step 1: Preparation of 1-(3-chloropropyl)-1H-1,2,4-triazole

1 g of a 1,2,4-triazole sodium salt was dissolved in N,N-dimethylformamide, and the solution was cooled to 0° C., to which 570 mg of 60% sodium hydride (NaH) was then added. The reactants were stirred for 20 min, and 1.3 mL of 1-bromo-3-chloropropane was slowly added thereto. The reaction mixture was warmed to room temperature and stirred for 12 hours, followed by addition of water and extraction with ethyl acetate. The organic layer was dried over anhydrous magnesium sulfate and concentrated under reduced pressure. The residue was purified by column chromatography to afford 600 mg (38%) of the title compound.

¹H NMR (CDCl₃): δ 8.08 (s, 1H), 7.93 (s, 1H), 4.37-4.34 (m, 2H), 3.47-3.43 (m, 2H), and 2.35-2.29 (m, 2H)

### Step 2: Preparation of cis-4-amino-5-chloro-N-[1-(3-(1H-1,2,4-triazol-1-yl)propyl)-3-methoxypiperidin-4-yl]-2-methoxybenzamide

1 g of cis-norcisapride was dissolved in N,N-dimethylformamide to which 600 mg of 1-(3-chloropropyl)-1H-1,2,4-triazole, 660 mg of potassium carbonate, and 106 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 610 mg (45%) of the title compound.

¹H NMR (CDCl₃): δ 8.16 (d, J=8.4 Hz, 1H), 8.11 (s, 1H), 8.06 (s, 1H), 7.91 (s, 1H), 6.27 (s, 1H), 4.37 (bs, 2H), 4.30-4.10 (m, 3H), 3.87 (s, 3H), 3.46-3.39 (m, 4H), 2.97-2.85 (m, 1H), 2.77-2.65 (m, 1H), 2.32-2.00 (m, 6H), and 1.92-1.68 (m, 2H)

[α]²⁵_{D}=−0.3 (c=0.5, MeOH)

### Example 47

### Preparation of (3S,4R)-4-amino-5-chloro-N-[1-(3-(1H-1,2,4-triazol-1-yl)propyl)-3-methoxypiperidin-4-yl]-2-methoxybenzamide

Analogously to Example 46, 400 mg of the title compound was prepared from 500 mg of a 1,2,4-triazole sodium salt, 0.65 mL of 1-bromo-3-chloropropane, and 500 mg of (+)-norcisapride.

[α]²⁵_{D}=−0.5 (c=0.5, MeOH)

### Example 48

### Preparation of cis-4-amino-5-chloro-N-[1-(3-(1H-tetrazol-2-yl)propyl)-3-methoxypiperidin-4-yl]-2-methoxybenzamide

Analogously to Example 46, 1.06 g of the title compound was prepared from 1 g of 1H-tetrazole, 1.18 mL of 1-bromo-3-chloropropane, and 1.78 g of cis-norcisapride.

¹H NMR (CDCl₃): δ 8.80 (s, 1H), 8.15 (d, J=8.0 Hz, 1H), 8.06 (s, 1H), 6.29 (s, 1H), 4.56-4.48 (m, 2H), 4.40 (bs, 2H), 4.18-4.10 (m, 1H), 3.88 (s, 3H), 3.46 (s, 3H), 3.43-3.40 (m, 1H), 2.93-2.85 (m, 1H), 2.77-2.68 (m, 1H), 2.33-2.27 (m, 1H), 2.22-2.05 (m, 5H), and 1.84-1.78 (m, 2H)

### Example 49

### Preparation of cis-4-amino-5-chloro-N-[1-(3-(1H-1,2,3-triazol-1-yl)propyl)-3-methoxypiperidin-4-yl]-2-methoxybenzamide

Analogously to Example 46, 1.19 g of the title compound was prepared from 1.06 g of 1H-1,2,3-triazole, 1.27 mL of 1-bromo-3-chloropropane, and 1.8 g of cis-norcisapride.

¹H NMR (CDCl₃): δ 8.16 (d, J=8.4 Hz, 1H), 8.07 (s, 1H), 7.67 (s, 1H), 7.59 (s, 1H), 6.27 (s, 1H), 4.49-4.41 (m, 2H), 4.36 (bs, 2H), 4.20-4.11 (m, 1H), 3.87 (s, 3H), 3.43 (bs, 4H), 3.02-2.90 (m, 1H), 2.78-2.66 (m, 1H), 2.38-2.25 (m, 2H), 2.20-2.05 (m, 4H), and 1.92-1.76 (m, 2H)

### Example 50

### Preparation of cis-4-amino-5-chloro-N-[1-(3H-pyrrol-1-yl)propyl)-3-methoxypiperidin-4-yl]-2-methoxybenzamide

Analogously to Step 2 of Example 46, 905 mg of the title compound was prepared from 0.53 mL of 1-(3-bromopropyl)-pyrrole and 1 g of cis-norcisapride.

¹H NMR (CDCl₃): δ 8.18 (d, J=8.0 Hz, 1H), 8.07 (s, 1H), 6.64 (t, 2.4 Hz, 2H), 6.27 (s, 1H), 6.11 (t, J=2.4 Hz, 2H), 4.37 (bs, 2H), 4.21-4.11 (m, 1H), 4.02-3.84 (m, 5H), 3.42 (bs, 4H), 3.06-2.92 (m, 1H), 2.80-2.70 (m, 1H), 2.40-2.26 (m, 2H), 2.23-2.08 (m, 2H), and 2.03-1.70 (m, 4H)

### Example 51

### Preparation of cis-4-amino-5-chloro-N-[1-(3-(1H-pyrrol-1-yl)ethyl)-3-methoxypiperidin-4-yl]-2-methoxybenzamide

Analogously to Step 2 of Example 46, 355 mg of the title compound was prepared from 0.45 mL of 1-(2-bromoethyl)-pyrrole and 1 g of cis-norcisapride.

¹H NMR (CDCl₃): δ 8.18 (d, J=8.4 Hz, 1H), 8.07 (s, 1H), 6.68 (t, J=2.4 Hz, 2H), 6.27 (s, 1H), 6.12 (t, J=2.4 Hz, 2H), 4.37 (bs, 2H), 4.17-4.13 (m, 1H), 4.03 (t, J=6.8 Hz, 2H), 3.86 (s, 3H), 3.42-3.39 (m, 4H), 2.98-2.94 (m, 1H), 2.81-2.72 (m, 3H), 2.31-2.20 (m, 2H), and 1.92-1.76 (m, 2H)

### Example 52

### Preparation of cis-4-amino-5-chloro-N-[1-(2-(bicyclo[2.2.1]heptan-2-yl)ethyl)-3-methoxypiperidin-4-yl]-2-methoxybenzamide

### Step 1: Preparation of 2-(bicyclo[2.2.1]heptan-2-yl)ethanol

2 mL of 2-norbornane acetic acid was dissolved in tetrahydrofuran and the solution was cooled to 0, to which 577 mg of lithium aluminum hydride (LAH) was then added. The reactants were slowly warmed to room temperature and stirred for 2 hours. The reaction was then terminated with addition of water and 10% sodium hydroxide (NaOH solution). The reaction solution was filtered through celite and concentrated under reduced pressure. The residue was purified by column chromatography to afford 1.62 g (84%) of the title compound.

¹H NMR (CDCl₃): δ 3.61 (t, J=6.4 Hz, 2H), 2.18 (bs, 1H), 1.95 (bs, 1H), 1.61-1.27 (m, 7H), and 1.18-1.00 (m, 4H)

### Step 2: Preparation of 2-(2-bromoethyl)bicyclo[2.2.1]heptane

1.62 g of 2-(bicyclo[2.2.1]heptan-2-ypethanol was dissolved in dichloromethane and the solution was cooled to 0° C., to which 3.34 g of triphenylphosphine and 2.27 g of N-bromosuccinimide were then added. The reactants were slowly warmed to room temperature, stirred for 12 hours and concentrated under reduced pressure. The residue was purified by column chromatography to afford 2.20 g (93%) of the title compound.

¹H NMR (CDCl₃): δ 3.35 (t, J=7.2 Hz, 2H), 2.20 (bs, 1H), 1.95 (bs, 1H), 1.86-1.81 (m, 1H), 1.65-1.42 (m, 5H), 1.27-1.24 (m, 1H), 1.20-1.07 (m, 3H), and 1.03-0.95 (m, 1H)

### Step 3: Preparation of cis-4-amino-5-chloro-N-[1-(2-bicyclo[2.2.1]heptan-2-yl)ethyl)-3-methoxypiperidin-4-yl]-2-methoxybenzamide

300 mg of cis-norcisapride was dissolved in N,N-dimethylformamide to which 233 mg of 2-(2-bromoethyl)bicyclo[2.2.1]heptane, 185 mg of potassium carbonate, and 32 mg of potassium iodide were then sequentially added. The reaction mixture was stirred at 90° C. for 5 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 253 mg (61%) of the title compound.

¹H NMR (CDCl₃): δ 8.17 (d, J=8.4 Hz, 1H), 8.03 (s, 1H), 6.25 (s, 1H), 4.45 (bs, 2H), 4.16-4.06 (m, 1H), 3.81 (s, 3H), 3.38 (bs, 4H), 3.06-2.94 (m, 1H), 2.77-2.66 (m, 1H), 2.35-2.19 (m, 2H), 2.17-2.00 (m, 2H), 1.92-1.71 (m, 3H), 1.50-1.33 (m, 4H), 1.32-1.20 (m, 4H), and 1.14-0.93 (m, 4H)

### Example 53

### Preparation of cis-4-amino-5-chloro-N-[1-(benzofuran-2-ylmethyl)-3-methoxypiperidin-4-yl]-2-methoxybenzamide

1 g of cis-norcisapride was dissolved in methanol to which 466 mg of 2-benzofurancarboxaldehyde, 500 mg of sodium cyanoborohydride (NaBH₃CN), and 1 mL of acetic acid were then sequentially added. The reaction mixture was stirred under reflux for 2 hours. After being cooled to room temperature, the reaction solution was concentrated under reduced pressure and extracted with ethyl acetate. The organic layer was dried over anhydrous magnesium sulfate and concentrated under reduced pressure. The residue was purified by column chromatography to afford 177 mg (13%) of the title compound.

¹H NMR (CDCl₃): δ 8.15 (d, J=8.0 Hz, 1H), 8.06 (s, 1H), 7.51 (d, J=7.6 Hz, 1H), 7.44 (d, J=7.6 Hz, 1H), 7.25-7.16 (m, 2H), 6.58 (s, 1H), 6.24 (s, 1H), 4.38 (bs, 2H), 4.21-4.13 (m, 1H), 3.85-3.69 (m, 5H), 3.44-3.41 (m, 1H), 3.36 (s, 3H), 3.10-2.96 (m, 1H), 2.90-2.78 (m, 1H), 2.45-2.29 (m, 2H), 2.00-1.91 (m, 1H), and 1.87-1.78 (m, 1H)

### Example 54

### Preparation of cis-4-amino-5-chloro-N-[1-(2-(5-methyl-1,2,4-oxadiazol-3-yl)ethyl)-3-methoxypiperidin-4-yl]-2-methoxybenzamide

### Step 1: Preparation of cis-4-amino-5-chloro-N-[1-(2-cyanoethyl)-3-methoxypiperidin-4-yl]-2-methoxybenzamide

3 g of cis-norcisapride was dissolved in N,N-dimethylformamide to which 0.95 mL of 3-bromopropionitrile, 1.85 g of potassium carbonate, and 317 mg of potassium iodide were then sequentially added. The reaction mixture was stirred at 90° C. for 3 hours and cooled to room temperature, followed by addition of water and extraction with ethyl acetate. The organic layer was dried over anhydrous magnesium sulfate and concentrated under reduced pressure to afford 1.49 g (43%) of the title compound.

¹H NMR (CDCl₃): δ 8.16 (d, J=8 Hz, 1H), 8.05 (s, 1H), 6.27 (s, 1H), 4.39 (bs, 2H), 4.18-4.11 (m, 1H), 3.85 (s, 3H), 3.42 (bs, 4H), 3.08-3.03 (m, 1H), 2.80-2.72 (m, 3H), 2.52 (t, J=7.2 Hz, 2H), 2.32-2.55 (m, 2H), and 1.91-1.72 (m, 2H)

### Step 2: Preparation cis-4-amino-5-chloro-N-[1-(3-amino-3-(hydroxyimino)propyl)-3-methoxypiperidin-4-yl]-2-methoxybenzamide

1 g of cis-4-amino-5-chloro-N-[1-(2-cyanoethyl)-3-methoxypiperidin-4-yl]-2-methoxybenzamide was dissolved in ethanol to which 379 mg of hydroxylamine hydrochloride and 688 mg of sodium bicarbonate were then added. The reactants were stirred under reflux for 12 hours and distilled under reduced pressure. The residue was dissolved in dimethyl chloride and water (q.s.) was added to result in solidification. The resulting solids were filtered to give 840 mg (76%) of the title compound.

¹H NMR (DMSO): δ 8.70 (s, 1H), 8.08 (d, J=8.0 Hz, 1H), 7.72 (s, 1H), 6.48 (s, 1H), 5.97 (bs, 2H), 5.44 (bs, 2H), 4.02-3.92 (m, 1H), 3.85 (s, 3H), 3.40-3.28 (m, 4H), 2.96-2.82 (m, 1H), 2.65-2.42 (m, 3H), 2.26-2.05 (m, 4H), and 1.74-1.57 (m, 21-1)

### Step 3: Preparation of cis-4-amino-5-chloro-N-[1-(2-(5-methyl-1,2,4-oxadiazol-3-yl)ethyl)-3-methoxypiperidin-4-yl]-2-methoxybenzamide

570 mg of cis-4-amino-5-chloro-N-[1-(3-amino-3-(hydroxyimino)propyl)-3-methoxypiperidin-4-yl]-2-methoxybenzamide was dissolved in 1,4-dioxane to which 0.63 mL of N,N-dimethylacetamide dimethylacetal was then added. The reactants were stirred under reflux for 2 hours, cooled to room temperature and extracted with ethyl acetate. The organic layer was dried over anhydrous magnesium sulfate and concentrated under reduced pressure. The residue was purified by column chromatography to afford 122 mg (20%) of the title compound.

¹H NMR (CDCl₃): δ 8.17 (d, J=8 Hz, 1H), 8.07 (s, 1H), 6.27 (s, 1H), 4.35 (bs, 2H), 4.23-4.13 (m, 1H), 3.86 (s, 3H), 3.43-3.42 (m, 4H), 3.13-3.05 (m, 1H), 2.94-2.90 (m, 2H), 2.87-2.79 (m, 3H), 2.55 (s, 3H), 2.33-2.23 (m, 2H), and 1.90-1.80 (m, 2H)

[α]²⁵_{D}=−0.8 (c=0.5, MeOH)

### Example 55

### Preparation of (3S,4R)-4-amino-5-chloro-N-[1-(2-(5-methyl-1,2,4-oxadiazol-3-yl)ethyl)-3-methoxypiperidin-4-yl]-2-methoxybenzamide

Analogously to Example 54, 411 mg of the title compound was prepared from 2 g of (+)-norcisapride and 0.64 mL of 3-bromopropionitrile.

### Example 56

### Preparation of cis-4-amino-5-chloro-N-[1-(3-oxo-3-(quinolin-5-yl-amino)propyl)-3-methoxypiperidin-4-yl]-2-methoxybenzamide

### Step 1: Preparation of N-(quinolin-5-yl)acrylamide

500 mg of 5-aminoquinoline was dissolved in dichloromethane, and the solution was cooled to 0° C. Then, 0.98 mL of triethylamine was added followed by gradual addition of 0.42 mL of acryloyl chloride. The reaction mixture was stirred for 2 hours, followed by addition of water and extraction with dichloromethane. The organic layer was dried over anhydrous magnesium sulfate, concentrated under reduced pressure, and purified by column chromatography to afford 250 mg (36%) of the title compound.

¹H NMR (CDCl₃): δ 8.83 (bs, 1H), 8.29 (bs, 1H), 8.12 (d, J=8.0 Hz, 1H), 7.90 (d, J=7.2 Hz, 1H), 7.682 (bs, 1H), 7.60-7.56 (m, 1H), 7.28-7.25 (m, 1H), 6.48-6.30 (m, 2H), and 5.72 (d, J=9.2 Hz, 1H)

### Step 2: Preparation of cis-4-amino-5-chloro-N-[1-(3-oxo-3-(quinolin-5-yl-amino)propyl)-3-methoxypiperidin-4-yl]-2-methoxybenzamide

330 mg of cis-norcisapride was dissolved in ethanol to which 250 mg of N-(quinolin-5-yl)acrylamide was then added. The reaction mixture was stirred at room temperature for 12 hours, distilled under reduced pressure to remove ethanol, extracted with dichloromethane, dried over anhydrous magnesium sulfate and then distilled under reduced pressure. The residue was purified by column chromatography to afford 320 mg (60%) of the title compound.

¹H NMR (CDCl₃): δ 11.18 (s, 1H), 8.90 (dd, J=4 Hz, 1.6 Hz, 1H), 8.59 (d, J=8.4, 1H), 8.20-8.17 (m, 2H), 8.07 (s, 1H), 7.88 (d, J=8.8 Hz, 1H), 7.69 (t, J=8 Hz, 1H), 7.37 (dd., J=8.8 Hz, 4.4 Hz, 1H), 6.28 (s, 1H), 4.44 (bs, 2H), 4.22-4.17 (m, 1H), 3.81 (s, 3H), 3.48 (bs, 1H), 3.46-3.35 (m, 1H), 3.11 (bs, 4H), 2.91-2.87 (m, 1H), 2.77-2.69 (m, 2H), 2.67-2.60 (m, 1H), 2.40-2.28 (m, 1H), 2.20-2.14 (m, 1H), and 2.02-1.95 (m, 2H)

### Example 57

### Preparation of cis-4-amino-5-chloro-N-[1-(3-oxo-3-(quinolin-6-yl)amino)propyl)-3-methoxypiperidin-4-yl]-2-methoxybenzamide

Analogously to Example 56, 288 mg of the title compound was prepared from 420 mg of 6-aminoquinoline, 0.35 mL of acryloyl chloride, and 400 mg of cis-norcisapride.

¹H NMR (CDCl₃): δ 11.74 (s, 1H), 8.77 (dd, J=4.0 Hz, 1.6 Hz, 1H), 8.61 (d, J=1.6, 1H), 8.31 (d, J=8.4 Hz, 1H), 8.12 (d, J=8.0 Hz, 1H), 8.09 (s, 1H), 7.98 (d, J=8.8 Hz, 1H), 7.67 (dd, J=8.8 Hz, 2.4 Hz, 1H), 7.35 (dd, J=8.0 Hz, 4 Hz, 1H), 6.32 (s, 1H), 4.78 (bs, 2H), 4.28-4.16 (m, 1H), 3.92 (s, 3H), 3.55 (bs, 1H), 3.38-3.32 (m, 4H), 3.13-3.04 (m, 1H), 2.86-2.72 (m, 2H), 2.61-2.58 (m, 2H), 2.36-2.18 (m, 2H), and 2.02-1.98 (m, 2H)

### Example 58

### Preparation of cis-4-amino-5-chloro-N-[1-(6-oxo-6-(quinolin-5-yl)amino)hexyl)-3-methoxypiperidin-4-yl]-2-methoxybenzamide

### Step 1: Preparation of 6-bromo-N-(quinolin-5-yl)hexanamide

836 mg of 5-aminoquinoline was dissolved in dichloromethane, and the solution was cooled to 0° C. Then, 1.22 mL of triethylamine was added followed by gradual addition of 1.05 mL of 6-bromohexanoyl chloride. The reaction mixture was stirred for 4 hours, followed by addition of water and extraction with dichloromethane. The organic layer was dried over anhydrous magnesium sulfate, concentrated under reduced pressure and then purified by column chromatography to afford 1.20 g (65%) of the title compound.

¹H NMR (CDCl₃): δ 8.85 (bs, 1H), 8.11 (d, J=8.4 Hz), 7.95-7.88 (m, 2H), 7.68-7.57 (m, 2H), 7.34-7.28 (m, 1H), J=6.8 Hz, 2H), 2.44 (t, J=7.2 Hz, 2H), 1.88-1.84 (m, 2H), 1.77-1.73 (m, 2H), and 1.53-1.49 (m, 2H)

### Step 2: Preparation of cis-4-amino-5-chloro-N-[1-(6-oxo-6-(quinolin-5-yl)amino)hexyl)-3-methoxypiperidin-4-yl]-2-methoxybenzamide

200 mg of cis-norcisapride was dissolved in N,N-dimethylformamide to which 246 mg of 6-bromo-N-(quinolin-5-yl)hexanamide, 123 mg of potassium carbonate, and 21 mg of potassium iodide were then sequentially added. The reaction mixture was stirred at 80° C. for 2 hours and cooled to room temperature, followed by addition of water and extraction with ethyl acetate. The organic layer was dried over anhydrous magnesium sulfate and concentrated under reduced pressure. The residue was purified by column chromatography to afford 64 mg (18%) of the title compound.

¹H NMR (CDCl₃): δ 8.89 (s, 1H), 8.25 (d, J=8.4 Hz, 1H), 8.16 (d, J=8.0 Hz, 1H), 8.05 (s, 1H), 7.96 (d, J=8.4 Hz, 1H), 7.81 (d, J=7.6 Hz, 1H), 7.68 (t, J=8.0 Hz, 1H), 7.40 (q, J=4 Hz, 1H), 6.25 (s, 1H), 4.37 (bs, 2H), 4.16-4.02 (m, 1H), 3.80 (s, 3H), 3.39 (bs, 4H), 3.16-2.96 (m, 1H), 2.87-2.70 (m, 1H), 2.60-2.30 (m, 4H), 2.20-1.95 (m, 3H), 1.93-1.68 (m, 4H), 1.67-1.53 (m, 2H), and 1.52-1.36 (m, 2H)

### Example 59

### Preparation of cis-4-amino-5-chloro-N-[1-(3-oxo-3-(4,6-dimethylpyridin-2-ylamino)propyl)-3-methoxypiperidin-4-yl]-2-methoxybenzamide

Analogously to Example 56, 78 mg of the title compound was prepared from 0.61 g of 2-amino-4,6-dimethylpyridine, 0.61 mL of acryloyl chloride, and 400 mg of cis-norcisapride.

¹H NMR (CDCl₃): δ 9.90 (bs, 1H), 8.19 (d, J=8.0 Hz, 1H), 8.08 (s, 1H), 7.77 (s, 1H), 6.69 (s, 1H), 6.27 (s, 1H), 4.37 (bs, 2H), 4.30-4.20 (m, 1H), 3.87 (s, 3H), 3.56-3.49 (m, 1H), 3.40 (s, 3H), 3.10-2.96 (m, 1H), 2.88-2.70 (m, 3H), 2.60-2.51 (m, 2H), 2.42-2.27 (m, 7H), 2.14-2.02 (m, 1H), and 1.94-1.76 (m, 2H)

### Example 60

### Preparation of cis-4-amino-5-chloro-N-[1-(6-oxo-6-(4,6-dimethylpyridin-2-ylamino)hexyl)-3-methoxypiperidin-4-yl]-2-methoxybenzamide

Analogously to Example 58, 130 mg of the title compound was prepared from 96 mg of 2-amino-4,6-dimethylpyridine, 0.14 mL of 6-bromohexanoyl chloride, and 200 mg of cis-norcisapride.

¹H NMR (CDCl₃): δ 8.19 (d, J=8.8 Hz, 1H), 8.07 (s, 1H), 7.83 (s, 1H), 7.78 (s, 1H), 6.70 (s, 1H), 6.27 (s, 1H), 4.36 (bs, 2H), 4.22-4.12 (m, 1H), 3.85 (s, 3H), 3.41 (bs, 4H), 3.10-2.96 (m, 1H), 2.83-2.72 (m, 1H), 2.47-2.29 (m, 9H), 2.24-2.08 ((m, 3H), 1.95-1.68 (m, 4H), 1.62-1.48 (m, 2H), and 1.43-1.32 (m, 2H)

### Example 61

### Preparation of cis-4-amino-5-chloro-N-[1-(3-oxo-3-(1H-indol-5-yl)amino)propyl)-3-methoxypiperidin-4-yl]-2-methoxybenzamide

Analogously to Example 56, 180 mg of the title compound was prepared from 429 mg of 5-aminoindole, 0.40 mL of acryloyl chloride, and 200 mg of cis-norcisapride.

¹H NMR (CDCl₃): δ 11.06 (s, 1H), 8.37 (s, 1H), 8.27 (d, J=8.0 Hz, 1H), 8.09 (s, 1H), 7.99 (d, J=1.2 Hz, 1H), 7.43 (dd, J=8.8 Hz, 1.6 Hz, 1H), 7.29 (d, J=8.8 Hz, 1H), 7.16 (t, J=2.4 Hz, 1H), 6.47 (t, J=2.4 Hz, 1H), 6.29 (s, 1H), 4.41 (bs, 2H), 4.24-4.16 (m, 1H), 3.90 (s, 3H), 3.52-3.48 (m, 1H), 3.37 (s, 3H), 3.35-3.25 (m, 1H), 3.06-2.95 (m, 1H), 2.80-2.68 (m, 2H), 2.60-2.53 (m, 2H), 2.32-2.15 (m, 2H), and 2.05-1.90 (m, 2H)

### Example 62

### Preparation of cis-4-amino-5-chloro-N-[1-(6-oxo-6-(1H-indol-5-yl)amino)hexyl)-3-methoxypiperidin-4-yl]-2-methoxybenzamide

Analogously to Example 58, 130 mg of the title compound was prepared from 107 mg of 5-aminoindole, 0.15 mL of 6-bromohexanoyl chloride, and 200 mg of cis-norcisapride.

¹H NMR (CDCl₃): δ 8.37-8.33 (m, 1H), 8.21 (d, J=−8.0 Hz, 1H), 8.08 (s, 1H), 7.82 (s, 1H), 7.39-7.34 (m, 1H), 7.32-7.16 (m, 3H), 6.47 (bs, 1H), 6.26 (s, 1H), 4.36 (bs, 2H), 4.20-4.10 (m, 1H), 3.83 (s, 3H), 3.49-3.36 (m, 6H), 3.08-2.96 (m, 1H), 2.79-2.70 (m, 1H), 2.40-2.25 (m, 2H), 2.24-1.92 (m, 2H), 1.88-1.66 (m, 2H), 1.60-1.00 (m, 4H), and 0.90-0.78 (m, 2H)

### Example 63

### Preparation of 4-[cis-4-(4-amino-5-chloro-2-methoxybenzoylamino)-3-methoxy-piperidin-1-ylmethyl]-piperidine-1-carboxylic acid isopropylamide

### Step 1: Preparation of 4-hydroxymethyl-piperidine-1-carboxylic acid isopropylamide

2 g of 4-piperidinemethanol was dissolved in dichloromethane, and the solution was cooled to 0° C., followed by slow addition of 1.8 mL of isopropyl isocyanate. The reaction mixture was warmed to room temperature, stirred for 3 hours, and concentrated under reduced pressure to remove dichloromethane. The residue was purified by column chromatography to afford 3.48 g (100%) of the title compound.

¹H NMR (CDCl₃): δ 4.23-4.12 (m, 1H), 3.97-3.91 (m, 3H), 3.51-3.48 (m, 2H), 2.74 (td, J=12.8 Hz, 2.8 Hz, 2H), 1.76-1.61 (m, 3H), 1.41-1.34 (m, 1H), and 1.22-1.12 (m, 8H)

### Step 2: Preparation of (1-(isopropylcarbamoyl)piperidin-4-yl)methyl methanesulfonate

3.48 g of 4-hydroxymethyl-piperidine-1-carboxylic acid isopropylamide was dissolved in dichloromethane, and the solution was cooled to 0° C. Then, 2.7 mL of triethylamine and 1.5 mL of methanesulfonyl chloride were added thereto. The reaction mixture was warmed to room temperature and stirred for 3 hours, followed by addition of water and extraction with dichloromethane. The extracted organic layer was dried over anhydrous magnesium sulfate, concentrated under reduced pressure, and purified by column chromatography to afford 3.76 g (78%) of the title compound.

¹H NMR (CDCl₃): δ 4.26-4.16 (m, 1H), 4.05 (d, J=6.4 Hz, 2H), 3.98-3.91 (m, 3H), 2.99 (s, 3H), 2.78-2.70 (m, 2H), 1.94-1.88 (m, 1H), 1.78-1.70 (m, 2H), 1.28-1.14 (m, 2H), and 1.12 (d, J=6.4 Hz, 6H)

### Step 3: Preparation of 4-[cis-4-(4-amino-5-chloro-2-methoxybenzoylamino)-3-methoxy-piperidin-1-ylmethyl]-piperidine-1-carboxylic acid isopropylamide

3.53 g of cis-norcisapride was dissolved in N,N-dimethylformamide to which 3.76 g of (1-(isopropylcarbamoyl)piperidin-4-yl)methyl methanesulfonate, 2.18 g of potassium carbonate, and 373 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 2.02 mg (36%) of the title compound.

¹H NMR (CDCl₃): δ 8.17 (d, J=8.0 Hz, 1H), 8.06 (s, 1H), 6.27 (s, 1H), 4.41 (bs, 2H), 4.24-4.13 (m, 2H), 3.96-3.82 (m, 6H), 3.39 (bs, 4H), 2.96-2.86 (m, 1H), 2.75-2.62 (m, 3H), 2.24-2.09 (m, 4H), 1.94-1.56 (m, 5H), and 1.18-1.05 (m, 8H)

### Example 64

### Preparation of 4-[cis-4-(4-amino-5-chloro-2-methoxybenzoylamino)-3-methoxy-piperidin-1-ylmethyl]-piperidine-1-carboxylic acid dimethylamide

### Step 1: Preparation of 4-hydroxymethyl-piperidine-1-carboxylic acid dimethylamide

1 g of 4-piperidinemethanol was dissolved in dichloromethane, and the solution was cooled to 0° C. Then, 2.56 mL of triethylamine and 0.84 mL of dimethylcarbamoyl chloride were added thereto. The reaction mixture was gradually warmed to room temperature and stirred for 3 hours, followed by addition of water and extraction with dichloromethane. The extracted organic layer was dried over anhydrous magnesium sulfate, concentrated under reduced pressure, and purified by column chromatography to afford 1.32 g (82%) of the title compound.

¹H NMR (CDCl₃): δ 3.71-3.63 (m, 2H), 3.49 (t, J=5.2 Hz, 2H), 2.79 (s, 6H), 2.76-2.69 (m, 2H), 1.75-1.58 (m, 3H), and 1.26-1.15 (m, 2H)

### Step 2: Preparation of 4-bromomethyl-piperidine-1-carboxylic acid dimethylamide

1.32 g of 4-hydroxymethyl-piperidine-1-carboxylic acid dimethylamide was dissolved in dichloromethane, and the solution was cooled to 0° C. Then, 2.04 g of triphenylphosphine and 1.39 g of N-bromosuccinimide were added thereto. The reaction mixture was warmed to room temperature, stirred for 12 hours and concentrated under reduced pressure. The residue was purified by column chromatography to afford 719 mg (41%) of the title compound.

¹H NMR (CDCl₃): δ 3.72-3.64 (m, 2H), 3.28 (t, J=5.2 Hz, 2H), 2.80 (s, 6H), 2.74-2.68 (m, 2H), 1.88-1.73 (m, 3H), and 1.32-1.17 (m, 2H)

### Step 3: Preparation of 4-[cis-4 (4-amino-5-chloro-2-methoxybenzoylamino)-3-methoxy-piperidin-1-ylmethyl]-piperidine-1-carboxylic acid dimethylamide

755 mg of cis-norcisapride was dissolved in N,N-dimethylformamide to which 719 mg of 4-bromomethyl-piperidine-1-carboxylic acid dimethylamide, 465 mg of potassium carbonate, 80 mg of potassium iodide were then sequentially added. The reaction mixture was stirred at 90° C. for 12 hours. After being cooled to room temperature, water was added to the reactants, followed by extraction with ethyl acetate. The organic layer was dried over anhydrous magnesium sulfate and concentrated under reduced pressure. The residue was purified by column chromatography to afford 310 mg (27%) of the title compound.

¹H NMR (CDCl₃): δ 8.16 (d, J=8.4 Hz, 1H), 8.05 (s, 1H), 6.26 (s, 1H), 4.42 (bs, 2H), 4.21-4.12 (m, 1H), 3.84 (s, 3H), 3.65-3.58 (m, 2H), 3.39 (bs, 4H), 2.95-2.84 (m, 1H), 2.78 (s, 6H), 2.73-2.60 (m, 3H), 2.22-2.08 (m, 4H), 2.02-1.58 (m, 5H), and 1.19-1.07 (m, 2H)

### Example 65

### Preparation of (3R,4S)-4-amino-5-chloro-N-[1-((1-isobutyrylpiperidin-4-yl)methyl)-3-methoxypiperidin-4-yl]-2-methoxybenzamide

Analogously to Example 14, 442 mg of the title compound was prepared from 0.39 mL of isobutyric acid, 486 mg of 4-piperidinemethanol, and 400 mg of 4-amino-5-chloro-2-methoxy-N-((3R,4S)-3-methoxypiperidin-4-yl)benzamide (hereinafter, referred to as “(−)-norcisapride”).

[α]²⁵_{D}=−11.2 (c=0.5, MeOH)

### Example 66

### Preparation of cis-4-amino-5-chloro-N-[1-((1-isobutyrylpiperidin-4-yl)methyl)-3-methoxypiperidin-4-yl]-2-methoxybenzamide hydrochloride

481 mg of cis-4-amino-5-chloro-N-[1-((1-isobutyrylpiperidin-4-yl)methyl)-3-methoxypiperidin-4-yl]-2-methoxybenzamide was dissolved in isopropyl alcohol to which 85 μl of 12N-hydrochloride (aq.) was then added at 0° C. The reaction mixture was stirred for 2 hours and filtered to afford 516 mg of the title compound.

¹H NMR (D₂O): δ 7.52 (s, 1H), 6.35 (s, 1H), 4.30-4.24 (m, 1H), 4.08-199 (m, 1H), 3.98-3.90 (m, 1H), 3.78-3.64 (m, 5H), 3.34 (bs, 4H), 3.04-2.79 (m, 6H), 2.57-2.51 (m, 1H), 2.13-2.02 (m, 1H), 1.98-1.83 (m, 2H), 1.70-1.57 (m, 2H), 1.20-0.96 (m, 2H), and 0.87 (t, J=6.8 Hz, 6H)

### Example 67

### Preparation of cis-4-amino-5-chloro-N-[1-((1-isobutyrylpiperidin-4-yl)methyl)-3-methoxypiperidin-4-yl]-2-methoxybenzamide maleate

481 mg of cis-4-amino-5-chloro-N-[1-((1-isobutyrylpiperidin-4-yl)methyl)-3-methoxypiperidin-4-yl]-2-methoxybenzamide was dissolved in isopropyl alcohol to which 116 mg of maleic acid was then added. The reaction mixture was stirred for 2 hours and filtered to afford 486 mg of the title compound.

¹H NMR (D₂O): δ 7.53 (s, 1H), 6.36 (s, 1H), 6.08 (s, 2H), 4.32-4.24 (m, 1H), 4.08-4.01 (m, 1H), 3.98-3.90 (m, 1H), 3.35 (bs, 4H), 3.08-2.78 (m, 6H), 2.57-2.54 (m, 1H), 2.14-2.02 (m, 1H), 1.98-1.84 (m, 2H), 1.70-1.58 (m, 2H), 1.22-0.98 (m, 2H), and 0.87 (t, J=6.8 Hz, 6H)

### Example 68

### Preparation of cis-4-amino-5-chloro-N-[1-((1-pivaloylpiperidin-4-yl)methyl)-3-methoxypiperidin-4-yl]-2-methoxybenzamide hydrochloride

Analogously to Example 66, 285 mg of the title compound was prepared from 300 mg of cis-4-amino-5-chloro-N-[1-((1-pivaloylpiperidin-4-yl)methyl)-3-methoxypiperidin-4-yl]-2-methoxybenzamide and 51 μl of 12N-hydrochloride (aq.) in acetone.

¹H NMR (D₂O): δ 7.55 (s, 1H), 6.44 (s, 1H), 4.28-1.18 (m, 2H), 4.08-4.02 (m, 1H), 3.80-3.71 (m, 5H), 3.40-3.31 (m, 4H), 3.06-2.86 (m, 6H), 2.14-2.05 (m, 1H), 1.98-1.84 (m, 2H), 1.66-1.58 (m, 2H), and 1.20-0.96 (m, 11H)

### Example 69

### Preparation of cis-4-amino-5-chloro-N-[1-((1-pivaloylpiperidin-4-yl)methyl)-3-methoxypiperidin-4-yl]-2-methoxybenzamide maleate

Analogously to Example 67, 290 mg of the title compound was prepared from 300 mg of cis-4-amino-5-chloro-N-[1-((1-pivaloylpiperidin-4-yl)methyl)-3-methoxypiperidin-4-yl]-2-methoxybenzamide and 70 mg of maleic acid in acetone.

¹H NMR (D₂O): δ 7.52 (s, 1H), 6.35 (s, 1H), 6.07 (s, 2H), 4.30-4.18 (m, 2H), 4.06-4.00 (m, 1H), 3.80-3.65 (m, 5H), 3.34 (bs, 4H), 3.06-2.84 (m, 6H), 2.14-2.06 (m, 1H), 1.98-1.84 (m, 2H), 1.68-1.58 (m, 2H), and 1.16-0.98 (m, 11H)

### Example 70

### Preparation of 4-[cis-4-(4-amino-5-chloro-2-methoxybenzoylamino)-3-methoxy-piperidin-1-ylmethyl]-piperidine-1-carboxylic acid isopropylamide hydrochloride

Analogously to Example 66, 203 mg of the title compound was prepared from 200 mg of 4-[cis-4-(4-amino-5-chloro-2-methoxybenzoylamino)-3-methoxy-piperidin-1-ylmethyl]-piperidine-1-carboxylic acid isopropylamide and 34 μl of 12N-hydrochloride (aq.) in acetone.

¹H NMR (D₂O): δ 7.52 (s, 1H), 6.41 (s, 1H), 4.05-3.99 (m, 1H), 3.80-3.59 (m, 8H), 3.38-3.30 (m, 4H), 3.05-2.84 (m, 4H), 2.70-2.62 (m, 2H), 2.02-1.84 (m, 3H), 1.60-1.50 (m, 2H), and 1.10-0.88 (m, 8H)

### Example 71

### Preparation of 4-[cis-4-(4-amino-5-chloro-2-methoxybenzoylamino)-3-methoxy-piperidin-1-ylmethyl]-piperidine-1-carboxylic acid isopropylamide maleate

Analogously to Example 67, 195 mg of the title compound was prepared from 200 mg of 4-[cis-4-(4-amino-5-chloro-2-methoxybenzoylamino)-3-methoxy-piperidin-1-ylmethyl]-piperidine-1-carboxylic acid isopropylamide and 47 mg of maleic acid in acetone.

¹H NMR (D₂O): δ 7.50 (s, 1H), 6.29 (s, 1H), 6.07 (s, 2H), 4.04-3.98 (m, 1H), 3.82-3.60 (m, 8H), 3.35 (bs, 4H), 3.06-2.84 (m, 4H), 2.73-2.62 (m, 2H), 2.02-1.85 (m, 3H), 1.60-1.52 (m, 2H), and 1.12-0.90 (m, 8H)

### Example 72

### Binding Affinity Compounds for 5-HT₄ Receptor

The binding affinity of the compounds for a human 5-HT₄ receptor was assayed according to the method as disclosed in the literature [Wyngaert et al., Journal of Neurochemistry, (1997) 69, 1810-1819]. For this purpose, COS-7 cells expressing the human 5-HT₄ receptor were constructed and lysed to obtain cell membrane lysates which were then used in binding assay experiments. For binding assay, the membrane lysates were mixed and incubated with different concentrations of test materials and [H3]-AGR113808. Each concentration of the test materials was set to 4 μM, 1 μM, 0.25 μM, and 0.0625 μM, respectively, whereas the concentration of [H3]-AGR113808 was set to 0.595 nM. After incubation was completed, the reaction products were collected in a GF/B glass fiber filter using a Packard cell harvester, and the bound radioactivity was then determined using a liquid cell scintillation counter (Packard TopCount NXT™, Perkin Elmer). Specific binding of the radioligand to the 5-HT₄ receptor was calculated by subtracting the non-specific binding of the radioligand from the total radioligand binding. IC₅₀ was calculated from % inhibition of specific binding of the radioligand to the 5-HT₄ receptor, with respect to varying concentrations of the test materials. The results thus obtained are given in Table 1 below.

TABLE 1

| Compounds | IC₅₀ (μM) |
|---|---|
| Cisapride | 0.370 |
| (1) | 0.23 |
| (2) | 0.083 |
| (3) | 0.151 |
| (5) | 0.13 |
| (6) | 0.094 |
| (7) | 0.053 |
| (8) | 0.13 |
| (9) | 0.108 |
| (10) | 0.093 |
| (11) | 0.03 |
| (12) | 0.192 |
| (13) | 0.146 |
| (14) | 0.141 |
| (20) | 0.106 |
| (22) | 0.073 |
| (23) | 0.061 |
| (24) | 0.058 |
| (25) | 0.078 |
| (26) | 0.045 |
| (27) | 0.089 |
| (29) | 0.034 |
| (30) | 0.108 |
| (31) | 0.214 |
| (33) | 0.107 |
| (34) | 0.052 |
| (35) | 0.148 |
| (36) | 0.175 |
| (37) | 0.175 |
| (38) | 0.217 |
| (39) | 0.047 |
| (42) | 0.047 |
| (43) | 0.167 |
| (44) | 0.116 |
| (56) | 0.011 |
| (57) | 0.053 |
| (58) | 0.077 |
| (59) | 0.14 |
| (60) | 0.085 |
| (61) | 0.048 |
| (62) | 0.044 |
| (63) | 0.025 |
| (64) | 0.029 |

As can be seen from Table 1, compounds of the present invention inhibited specific binding of the radioligand to the 5-HT₄ receptor at a lower concentration, as compared to cisapride as a control, thus representing that the inventive compounds have strong binding affinity for 5-HT₄.

### Experimental Example 1

### Acute Oral Toxicity of Compounds in Mice

In order to examine acute toxicity of compounds according to the present invention, the following experiment was carried out.

Each 200 mg of compounds of Examples 1, 2, 6, 14, 17, 29, 54, 56, 59, 63 and 64 was loaded into 1% hydroxypropylmethylcellulose matrix, and the resulting formulations were then orally administered to 5-week old male ICR mice (20 g±2 g, n=5) at a dose of 1 g/10 mL/kg. Over the entire experimental period of 2 weeks, the minimum lethal dose (MLD, mg/kg) of the compound was investigated by the observation of the mortality, body weight and clinical symptoms of animals. The results thus obtained are given in Table 2. Cisapride was employed as a control drug.

TABLE 2

| Compounds | Minimum lethal dose (MLD, mg/kg) |
|---|---|
| Cisapride | >1000 |
| 1 | >1000 |
| 2 | >1000 |
| 6 | >1000 |
| 14 | >1000 |
| 17 | >1000 |
| 29 | >1000 |
| 54 | >1000 |
| 56 | >1000 |
| 59 | >1000 |
| 63 | >1000 |
| 64 | >1000 |

As can be seen from the acute toxicity test results of Table 2, all of the compounds used in the test exhibited MLD of 1000 mg/kg or higher, thus representing that the inventive compounds are safe for use

### Experimental Example 2

### Binding Affinity of Compounds for hERG Receptor

The binding affinity for the human ether-a-go-go-related gene (hERG) potassium (K+) channel was assayed in MDS Pharma Service (Catalog No 265900). Membrane lysates were obtained from mammalian HEK-293 cells expressing the hERG potassium channel and used in binding assay experiments. For the binding assay experiment, the membrane lysates were mixed and incubated with 0.2 μM or 10 μM of test materials and 1.5 nM of [H3]-Astemizole. After incubation was completed, the radioactivity bound to the hERG K+ channel was counted. The affinity of each test material for the hERG K+ channel was calculated from % inhibition of specific binding of the radioligand to the hERG K+ channel, resulting from the action of the test material. The results thus obtained are given in Table 3 below.

TABLE 3

| Compounds | hERG binding affinity (% at 10 μM) |
|---|---|
| Cisapride | 58% at 0.2 μM |
| 14 | 14 |
| 26 | 38 |
| 29 | 28 |
| 55 | 31 |
| 56 | 23 |
| 63 | 31 |
| 64 | 10 |

Development of cardiac arrhythmia which is a fatal adverse effect of cisapride is due to the cardiac QT prolongation that results from excessively high affinity of the drug for the hERG receptor. From the experimental result data, cisapride at a dose of 0.2 μM exhibited binding affinity of 58% for the hERG receptor, thus representing a high probability to cause arrhythmia. On the other hand, the binding affinity of benzamide derivatives in accordance with the present invention for the hERG receptor was less than 50% at a dose of 10 μM which is 50-fold higher than the test concentration of cisapride, thus suggesting that the inventive benzamide derivative compounds can significantly reduce the risk of developing arrhythmia.

### INDUSTRIAL APPLICABILITY

As apparent from the above description, novel benzamide derivative compounds of the present invention minimize the pathogenic risk of cardiac arrhythmia and activate a 5-HT₄ receptor. That is, these benzamide derivatives provide various beneficial advantages such as pronounced affinity for 5-HT₄ receptors, capability to reduce the gastric evacuation time, alleviation of ventricular tachycardia, ventricular fibrillation, torsades de pointes and QT prolongation, and promising applicability as a therapeutic agent for digestive system diseases due to low toxicity.

1. A compound represented by formula 1: 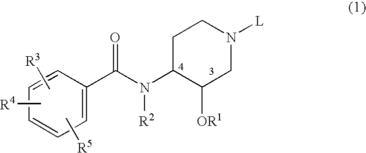
wherein:
R¹ is hydrogen or C₁₋₆ alkyl;
R² is hydrogen or C₁₋₆ alkyl;
R³, R⁴ and R⁵ are independently hydrogen, C₁₋₆ alkyl, C₁₋₆ alkoxy, amino, hydroxy, cyano, nitro, or halogen; and
L is 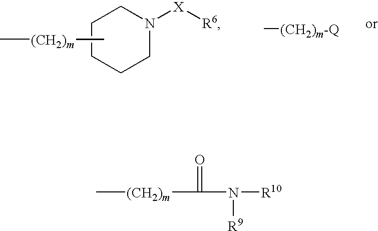
wherein m is an integer of 1 to 5; X is —(C═O)—, —(C═S)—, or —SO₂—; R⁶ is C₁₋₁₀ alkyl, C₁₋₁₀ alkenyl, C₁₋₁₀ alkoxy, C₁₋₁₀ thioalkoxy, or NR⁷R⁸ wherein R⁷ and R⁸, which are identical or different, are independently hydrogen or C₁₋₁₀ alkyl; Q is pyrrole, 1,2,3-triazole, 1,2,4-triazole, tetrazole, oxazole, isoxazole, thiazole, thiadiazole, oxadiazole, or benzofuran, each of which being optionally substituted by C₃₋₁₀ cycloalkyl, C₁₋₆ alkyl or nitro; and R⁹ and R¹⁰, which are identical or different, are independently pyridine, indole, or quinoline, each of which being optionally substituted by hydrogen or C₁₋₆ alkyl; or an isomer, a pharmaceutically acceptable salt or a hydrate thereof.

2. The compound according to claim 1, wherein the position 3 and position 4 in formula 1 are in a cis configuration, a (3S,4R) configuration, or a (3R,4S) configuration.

3. The compound according to claim 1, wherein R¹ is methyl.

4. The compound according to claim 1, wherein R² is hydrogen.

5. The compound according to claim 1, wherein R³, R⁴, and R⁵ are independently chloro, amino or methoxy.

6. The compound according to claim 5, wherein R³ is 2-methoxy, R⁴ is 4-amino, and R⁵ is chloro.

7. The compound according to claim 1, wherein L is

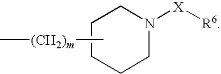

8. The compound according to claim 1, wherein L is

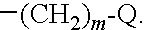

9. The compound according to claim 1, wherein L is

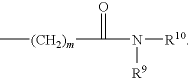

10. The compound according to claim 1, wherein the compound is selected from the group consisting of:
ethyl 4-[cis-4-(4-amino-5-chloro-2-methoxybenzamido)-3-methoxypiperidin-1-yl)methyl]piperidine-1-carboxylate,
ethyl 4-[((3S,4R)-4-(4-amino-5-chloro-2-methoxybenzamido)-3-methoxypiperidin-1-yl)methyl]piperidine-1-carboxylate,
ethyl 4-[2-(cis-4-(4-amino-5-chloro-2-methoxybenzamido)-3-methoxypiperidin-1-yl)ethyl]piperidine-1-carboxylate,
ethyl 2-[2-(cis-4-(4-amino-5-chloro-2-methoxybenzamido)-3-methoxypiperidin-1-yl)ethyl]piperidine-1-carboxylate,
methyl 4-[cis-4-(4-amino-5-chloro-2-methoxybenzamido)-3-methoxypiperidin-1-yl)methyl]piperidine-1-carboxylate,
propyl 4-[(cis-4-(4-amino-5-chloro-2-methoxybenzamido)-3-methoxypiperidin-1-yl)methyl]piperidine-1-carboxylate,
butyl 4-[cis-4-(4-amino-5-chloro-2-methoxybenzamido)-3-methoxypiperidin-1-yl)methyl]piperidine-1-carboxylate,
isopropyl 4-[cis-4-(4-amino-5-chloro-2-methoxybenzamido)-3-methoxypiperidin-1-yl)methyl]piperidine-1-carboxylate,
isobutyl 4-[cis-4-(4-amino-5-chloro-2-methoxybenzamido)-3-methoxypiperidin-1-yl)methyl]piperidine-1-carboxylate,
allyl 4-[(cis-4-(4-amino-5-chloro-2-methoxybenzamido)-3-methoxypiperidin-1-yl)methyl]piperidine-1-carboxylate,
2-ethylhexyl 4-[(cis-4-(4-amino-5-chloro-2-methoxybenzamido)-3-methoxypiperidin-1-yl)methyl]piperidine-1-carboxylate,
3-methyl-pentyl 4-[(cis-4-(4-amino-5-chloro-2-methoxybenzamido)-3-methoxypiperidin-1-yl)methyl]piperidine-1-carboxylate,
4-methyl-pentyl 4-[(cis-4-(4-amino-5-chloro-2-methoxybenzamido)-3-methoxypiperidin-1-yl)methyl]piperidine-1-carboxylate,
cis-4-amino-5-chloro-N-[1-((1-isobutyrylpiperidin-4-yl)methyl)-3-methoxypiperidin-4-yl]-2-methoxybenzamide,
(3S,4R)-4-amino-5-chloro-N-[1-((1-isobutyrylpiperidin-4-yl)methyl)-3-methoxypiperidin-4-yl]-2-methoxybenzamide,
cis-4-amino-5-chloro-N-[1-((1-isobutyrylpiperidin-4-yl)ethyl)-3-methoxypiperidin-4-yl]-2-methoxybenzamide,
cis-4-amino-5-chloro-N-[1-((1-propionylpiperidin-4-yl)methyl)-3-methoxypiperidin-4-yl]-2-methoxybenzamide,
cis-4-amino-5-chloro-N-[1-(2-(1-propionylpiperidin-4-yl)ethyl)-3-methoxypiperidin-4-yl]-2-methoxybenzamide,
cis-4-amino-5-chloro-N-[1-((1-propionylpiperidin-3-yl)methyl)-3-methoxypiperidin-4-yl]-2-methoxybenzamide,
cis-4-amino-5-chloro-N-[1-((1-butyrylpiperidin-4-yl)methyl)-3-methoxypiperidin-4-yl]-2-methoxybenzamide,
cis-4-amino-5-chloro-N-[1-(1-butyrylpiperidin-3-yl)methyl)-3-methoxypiperidin-4-yl]-2-methoxybenzamide,
cis-4-amino-5-chloro-N-[1-((1-pentanoylpiperidin-4-yl)methyl)-3-methoxypiperidin-4-yl]-2-methoxybenzamide,
cis-4-amino-5-chloro-N-[1-((1-hexanoylpiperidin-4-yl)methyl)-3-methoxypiperidin-4-yl]-2-methoxybenzamide,
cis-4-amino-5-chloro-N-[1-((1-(2-methylpentanoyl)piperidin-4-yl)methyl)-3-methoxypiperidin-4-yl]-2-methoxybenzamide,
cis-4-amino-5-chloro-N-[1-((1-(3-methylbutanoyl)piperidin-4-yl)methyl)-3-methoxypiperidin-4-yl]-2-methoxybenzamide,
cis-4-amino-5-chloro-N-[1-((1-(3,3-dimethylbutanoyl)piperidin-4-yl)methyl)-3-methoxypiperidin-4-yl]-2-methoxybenzamide,
cis-4-amino-5-chloro-N-[1-((1-(4-methylpentanoyl)piperidin-4-yl)methyl)-3-methoxypiperidin-4-yl]-2-methoxybenzamide,
cis-4-amino-5-chloro-N-[1-((1-acetylpiperidin-4-yl)methyl)-3-methoxypiperidin-4-yl]-2-methoxybenzamide,
cis-4-amino-5-chloro-N-[1-((1-pivaloylpiperidin-4-yl)methyl)-3-methoxypiperidin-4-yl]-2-methoxybenzamide,
cis-4-amino-5-chloro-N-[1-((1-(2-methylpropanethioyl)piperidin-4-yl)methyl)-3-methoxypiperidin-4-yl]-2-methoxybenzamide,
cis-4-amino-5-chloro-N-[1-((1-ethanethioylpiperidin-4-yl)methyl)-3-methoxypiperidin-4-yl]-2-methoxybenzamide,
cis-4-amino-5-chloro-N-[1-((1-propanethioylpiperidin-4-yl)methyl)-3-methoxypiperidin-4-yl]-2-methoxybenzamide,
cis-4-amino-5-chloro-N-[1-((1-pentanethioylpiperidin-4-yl)methyl)-3-methoxypiperidin-4-yl]-2-methoxybenzamide,
cis-4-amino-5-chloro-N-[1-((1-hexanethioylpiperidin-4-yl)methyl)-3-methoxypiperidin-4-yl]-2-methoxybenzamide,
cis-4-amino-5-chloro-N-[1-((1-butanethioylpiperidin-4-yl)methyl)-3-methoxypiperidin-4-yl]-2-methoxybenzamide,
cis-4-amino-5-chloro-N-[1-((1-(3-methylbutanethioyl)piperidin-4-yl)methyl)-3-methoxypiperidin-4-yl]-2-methoxybenzamide,
cis-4-amino-5-chloro-N-[1-((1-(4-methylpentanethioyl)piperidin-4-yl)methyl)-3-methoxypiperidin-4-yl]-2-methoxybenzamide,
cis-4-amino-5-chloro-N-[1-(1-(2,2-dimethylpropanethioyl)piperidin-4-yl)methyl)-3-methoxypiperidin-4-yl]-2-methoxybenzamide,
S-ethyl 4-[cis-4-(4-amino-5-chloro-2-methoxybenzamido)-3-methoxypiperidin-1-yl)methyl]piperidine-1-carbothioate,
S-propyl 4-[cis-4-(4-amino-5-chloro-2-methoxybenzamido)-3-methoxypiperidin-1-yl)methyl]piperidine-1-carbothioate,
S-butyl 4-[cis-4-(4-amino-5-chloro-2-methoxybenzamido)-3-methoxypiperidin-1-yl)methyl]piperidine-1-carbothioate,
cis-4-amino-5-chloro-N-[1-((1-(isopropylsulfonyl)piperidin-4-yl)methyl)-3-methoxypiperidin-4-yl]-2-methoxybenzamide,
cis-4-amino-5-chloro-N-[1-((1-(methylsulfonyl)piperidin-4-yl)methyl)-3-methoxypiperidin-4-yl]-2-methoxybenzamide,
(3S,4R)-4-amino-5-chloro-N-[1-(1-(methylsulfonyl)piperidin-4-yl)methyl)-3-methoxypiperidin-4-yl]-2-methoxybenzamide,
cis-4-amino-5-chloro-N-[1-(2-(1-(methylsulfonyl)piperidin-4-yl)ethyl)-3-methoxypiperidin-4-yl]-2-methoxybenzamide,
cis-4-amino-5-chloro-N-[1-(3-(1H-1,2,4-triazol-1-yl)propyl)-3-methoxypiperidin-4-yl]-2-methoxybenzamide,
(3S,4R)-4-amino-5-chloro-N-[1-(3-(1H-1,2,4-triazol-1-yl)propyl)-3-methoxypiperidin-4-yl]-2-methoxybenzamide,
cis-4-amino-5-chloro-N-[1-(3-(1H-tetrazol-2-yl)propyl)-3-methoxypiperidin-4-yl]-2-methoxybenzamide,
cis-4-amino-5-chloro-N-[1-(3-(1H-1,2,3-triazol-1-yl)propyl)-3-methoxypiperidin-4-yl]-2-methoxybenzamide,
cis-4-amino-5-chloro-N-[1-(3-(1H-pyrrol-1-yl)propyl)-3-methoxypiperidin-4-yl]-2-methoxybenzamide,
cis-4-amino-5-chloro-N-[1-(2-(1H-pyrrol-1-yl)ethyl)-3-methoxypiperidin-4-yl]-2-methoxybenzamide,
cis-4-amino-5-chloro-N-[1-(2-(bicyclo[2.2.1]heptan-2-yl)ethyl)-3-methoxypiperidin-4-yl]-2-methoxybenzamide,
cis-4-amino-5-chloro-N-[1-(benzofuran-2-ylmethyl)-3-methoxypiperidin-4-yl]-2-methoxybenzamide,
cis-4-amino-5-chloro-N-[1-(2-(5-methyl-1,2,4-oxadiazol-3-yl)ethyl)-3-methoxypiperidin-4-yl]-2-methoxybenzamide,
(3S,4R)-4-amino-5-chloro-N-[1-(2-(5-methyl-1,2,4-oxadiazol-3-yl)ethyl)-3-methoxypiperidin-4-yl]-2-methoxybenzamide,
cis-4-amino-5-chloro-N-[1-(3-oxo-3-(quinolin-5-ylamino)propyl)-3-methoxypiperidin-4-yl]-2-methoxybenzamide,
cis-4-amino-5-chloro-N-[1-(3-oxo-3-(quinolin-6-ylamino)propyl)-3-methoxypiperidin-4-yl]-2-methoxybenzamide,
cis-4-amino-5-chloro-N-[1-(6-oxo-6-(quinolin-5-ylamino)hexyl)-3-methoxypiperidin-4-yl]-2-methoxybenzamide,
cis-4-amino-5-chloro-N-[1-(3-oxo-3-(4,6-dimethylpyridin-2-ylamino)propyl)-3-methoxypiperidin-4-yl]-2-methoxybenzamide,
cis-4-amino-5-chloro-N-[1-(6-oxo-6-(4,6-dimethylpyridin-2-ylamino)hexyl)-3-methoxypiperidin-4-yl]-2-methoxybenzamide,
cis-4-amino-5-chloro-N-[1-(3-oxo-3-(1H-indol-5-ylamino)propyl)-3-methoxypiperidin-4-yl]-2-methoxybenzamide,
cis-4-amino-5-chloro-N-[1-(6-oxo-6-(1H-indol-5-ylamino)hexyl)-3-methoxypiperidin-4-yl]-2-methoxybenzamide,
4-[cis-4-(4-amino-5-chloro-2-methoxybenzoylamino)-3-methoxy-piperidin-1-ylmethyl]-piperidine-1-carboxylic acid isopropylamide,
4-[cis-4-(4-amino-5-chloro-2-methoxybenzoylamino)-3-methoxy-piperidin-1-ylmethyl]-piperidine-1-carboxylic acid dimethylamide,
(3R,4S)-4-amino-5-chloro-N-[1-((1-isobutyrylpiperidin-4-yl)methyl)-3-methoxypiperidin-4-yl]-2-methoxybenzamide,
cis-4-amino-5-chloro-N-[1-(1-isobutyrylpiperidin-4-yl)methyl)-3-methoxypiperidin-4-yl]-2-methoxybenzamide hydrochloride,
cis-4-amino-5-chloro-N-[1-(1-isobutyrylpiperidin-4-yl)methyl)-3-methoxypiperidin-4-yl]-2-methoxybenzamide maleate,
cis-4-amino-5-chloro-N-[1-(1-pivaloylpiperidin-4-yl)methyl)-3-methoxypiperidin-4-yl]-2-methoxybenzamide hydrochloride,
cis-4-amino-5-chloro-N-[1-(1-pivaloylpiperidin-4-yl)methyl)-3-methoxypiperidin-4-yl]-2-methoxybenzamide maleate,
4-[cis-4-(4-amino-5-chloro-2-methoxybenzoylamino)-3-methoxy-piperidin-1-ylmethyl]-piperidine-1-carboxylic acid isopropylamide hydrochloride, and
4-[cis-4-(4-amino-5-chloro-2-methoxybenzoylamino)-3-methoxy-piperidin-1-ylmethyl]-piperidine-1-carboxylic acid isopropylamide maleate.

11. A process for preparing a compound represented by formula 1, comprising:
(1) introducing a substituent at the amine of a compound of formula III to form a compound of formula IV;
(2) substituting hydroxy of the compound of formula IV by halogen or sulfonate to form a compound of formula V; and
(3) reacting the resulting compound of formula V with a piperidine-benzamide compound of formula II to prepare a compound of formula 1-1: 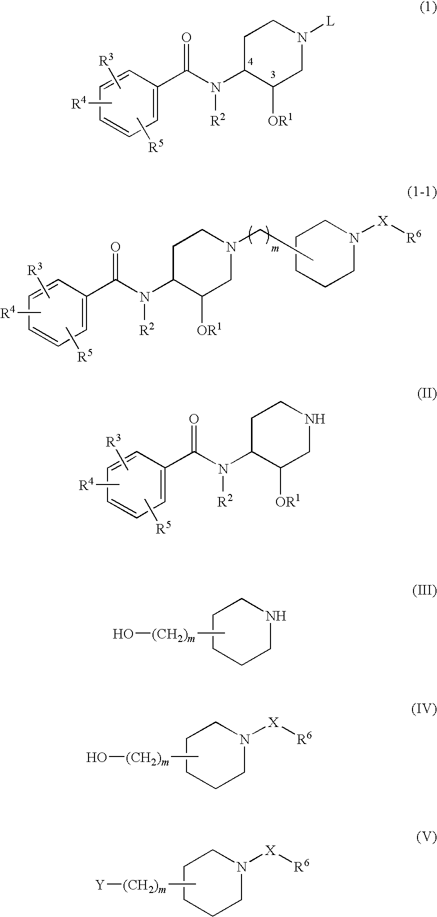
wherein L, X, R¹, R², R³, R⁴, R⁵, R⁶, and m are as defined in claim 1, and Y is a halogen atom or sulfonate.

12. A process for preparing a compound represented by formula 1, comprising:
(1) substituting Y² of a compound of formula VI with Q to form a compound of formula VII; and
(2) reacting the resulting compound of formula VII with a piperidine-benzamide compound of formula II to prepare a compound of formula 1-2: 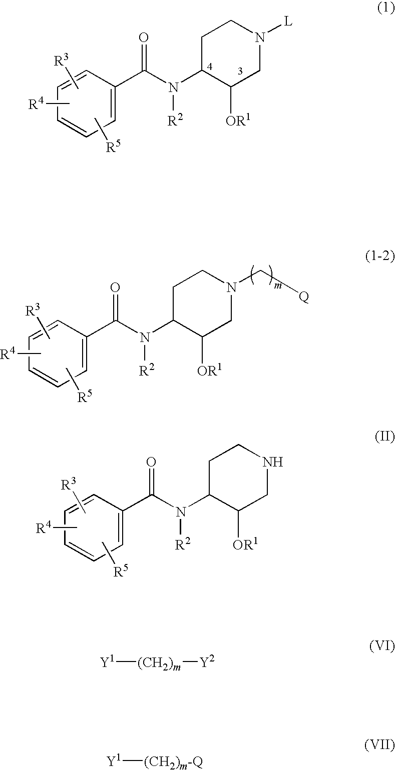
wherein L, R¹, R², R³, R⁴, R⁵, R⁶, m and Q are as defined in claim 1, and Y¹ and Y² are independently a halogen atom.

13. A process for preparing a compound represented by formula 1, comprising:
(1) reacting an acid chloride compound of formula VIII or XI with an amine compound of formula X to form an amide compound of formula IX or XII, and
(2) reacting the resulting compound of formula IX or XII with a piperidine-benzamide compound of formula II to prepare a compound of formula 1-3: 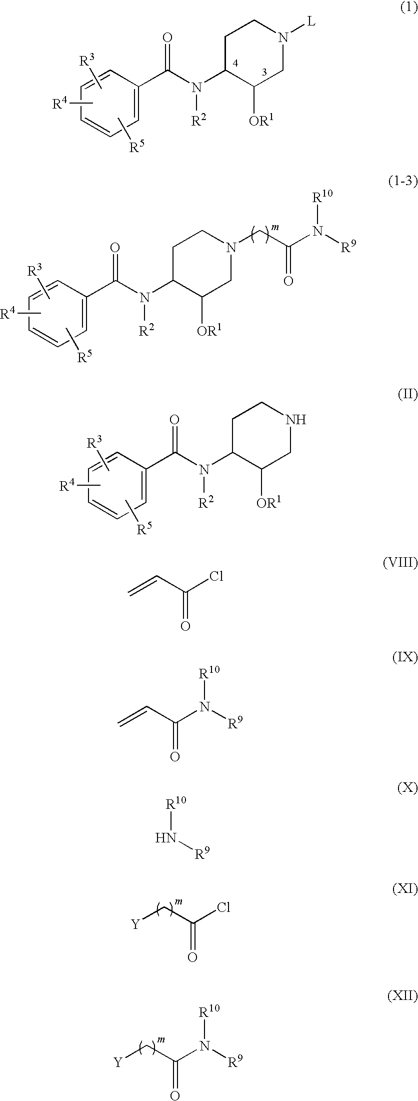
wherein L, R¹, R², R³, R⁴, R⁵, R⁹, R¹⁰, and m are as defined in claim 1, and Y is a halogen atom.

14. A composition for activating a 5-HT₄ receptor, comprising a compound represented by formula 1, or an isomer, a pharmaceutically acceptable salt or a hydrate thereof, as an active ingredient. 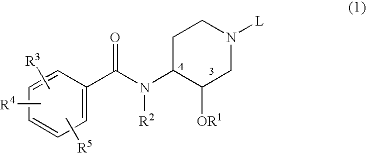
wherein:
R¹ is hydrogen or C₁₋₆ alkyl;
R² is hydrogen or C₁₋₆ alkyl;
R³, R⁴ and R⁵ are independently hydrogen, C₁₋₆ alkyl, C₁₋₆ alkoxy, amino, hydroxy, cyano, nitro, or halogen; and
L is 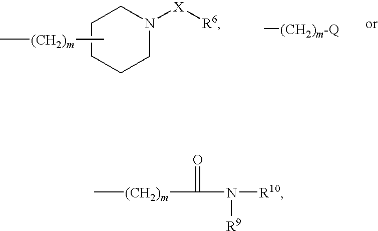
wherein m is an integer of 1 to 5; X is —(C═O)—, —(C═S)—, or —SO₂—; R⁶ is C₁₋₁₀ alkyl, C₁₋₁₀ alkenyl, C₁₋₁₀ alkoxy, C₁₋₁₀ thioalkoxy, or NR⁷R⁸ wherein R⁷ and R⁸, which are identical or different, are independently hydrogen or C₁₋₁₀ alkyl; Q is pyrrole, 1,2,3-triazole, 1,2,4-triazole, tetrazole, oxazole, isoxazole, thiazole, thiadiazole, oxadiazole, or benzofuran, each of which being optionally substituted by C₃₋₁₀ cycloalkyl, C₁₋₆ alkyl or nitro; and R⁹ and R¹⁰, which are identical or different, are independently pyridine, indole, or quinoline, each of which being optionally substituted by hydrogen or C₁₋₆ alkyl.

15. The composition according to claim 14, wherein the compound is selected from the group consisting of:
ethyl 4-[cis-4-(4-amino-5-chloro-2-methoxybenzamido)-3-methoxypiperidin-1-yl)methyl]piperidine-1-carboxylate,
ethyl 4-[((3S,4R)-4-(4-amino-5-chloro-2-methoxybenzamido)-3-methoxypiperidin-1-yl)methyl]piperidine-1-carboxylate,
ethyl 4-[2-(cis-4-(4-amino-5-chloro-2-methoxybenzamido)-3-methoxypiperidin-1-yl)ethyl]piperidine-1-carboxylate,
ethyl 2-[2-(cis-4-(4-amino-5-chloro-2-methoxybenzamido)-3-methoxypiperidin-1-yl)ethyl]piperidine-1-carboxylate,
methyl 4-[(cis-4-(4-amino-5-chloro-2-methoxybenzamido)-3-methoxypiperidin-1-yl)methyl]piperidine-1-carboxylate,
propyl 4-[(cis-4-(4-amino-5-chloro-2-methoxybenzamido)-3-methoxypiperidin-1-yl)methyl]piperidine-1-carboxylate,
butyl 4-[cis-4-(4-amino-5-chloro-2-methoxybenzamido)-3-methoxypiperidin-1-yl)methyl]piperidine-1-carboxylate,
isopropyl 4-[cis-4-(4-amino-5-chloro-2-methoxybenzamido)-3-methoxypiperidin-1-yl)methyl]piperidine-1-carboxylate,
isobutyl 4-[cis-4-(4-amino-5-chloro-2-methoxybenzamido)-3-methoxypiperidin-1-yl)methyl]piperidine-1-carboxylate,
allyl 4-[(cis-4-(4-amino-5-chloro-2-methoxybenzamido)-3-methoxypiperidin-1-yl)methyl]piperidine-1-carboxylate,
2-ethylhexyl 4-[(cis-4-(4-amino-5-chloro-2-methoxybenzamido)-3-methoxypiperidin-1-yl)methyl]piperidine-1-carboxylate,
3-methyl-pentyl 4-[(cis-4-(4-amino-5-chloro-2-methoxybenzamido)-3-methoxypiperidin-1-yl)methyl]piperidine-1-carboxylate,
4-methyl-pentyl 4-[(cis-4-(4-amino-5-chloro-2-methoxybenzamido)-3-methoxypiperidin-1-yl)methyl]piperidine-1-carboxylate,
cis-4-amino-5-chloro-N-[1-((1-isobutyrylpiperidin-4-yl)methyl)-3-methoxypiperidin-4-yl]-2-methoxybenzamide,
(3S,4R)-4-amino-5-chloro-N-[1-((1-isobutyrylpiperidin-4-yl)methyl)-3-methoxypiperidin-4-yl]-2-methoxybenzamide,
cis-4-amino-5-chloro-N-[1-(2-(1-isobutyrylpiperidin-4-yl)ethyl)-3-methoxypiperidin-4-yl]-2-methoxybenzamide,
cis-4-amino-5-chloro-N-[1-((1-propionylpiperidin-4-yl)methyl)-3-methoxypiperidin-4-yl]-2-methoxybenzamide,
cis-4-amino-5-chloro-N-[1-(2-(1-propionylpiperidin-4-yl)ethyl)-3-methoxypiperidin-4-yl]-2-methoxybenzamide,
cis-4-amino-5-chloro-N-[1-((1-propionylpiperidin-3-yl)methyl)-3-methoxypiperidin-4-yl]-2-methoxybenzamide,
cis-4-amino-5-chloro-N-[1-((1-butyrylpiperidin-4-yl)methyl)-3-methoxypiperidin-4-yl]-2-methoxybenzamide,
cis-4-amino-5-chloro-N-[1-((1-butyrylpiperidin-3-yl)methyl)-3-methoxypiperidin-4-yl]-2-methoxybenzamide,
cis-4-amino-5-chloro-N-[1-((1-pentanoylpiperidin-4-yl)methyl)-3-methoxypiperidin-4-yl]-2-methoxybenzamide,
cis-4-amino-5-chloro-N-[1-((1-hexanoylpiperidin-4-yl)methyl)-3-methoxypiperidin-4-yl]-2-methoxybenzamide,
cis-4-amino-5-chloro-N-[1-((1-(2-methylpentanoyl)piperidin-4-yl)methyl)-3-methoxypiperidin-4-yl]-2-methoxybenzamide,
cis-4-amino-5-chloro-N-[1-((1-(3-methylbutanoyl)piperidin-4-yl)methyl)-3-methoxypiperidin-4-yl]-2-methoxybenzamide,
cis-4-amino-5-chloro-N-[1-((1-(3,3-dimethylbutanoyl)piperidin-4-yl)methyl)-3-methoxypiperidin-4-yl]-2-methoxybenzamide,
cis-4-amino-5-chloro-N-[1-((1-(4-methylpentanoyl)piperidin-4-yl)methyl)-3-methoxypiperidin-4-yl]-2-methoxybenzamide,
cis-4-amino-5-chloro-N-[1-((1-acetylpiperidin-4-yl)methyl)-3-methoxypiperidin-4-yl]-2-methoxybenzamide,
cis-4-amino-5-chloro-N-[1-((1-pivaloylpiperidin-4-yl)methyl)-3-methoxypiperidin-4-yl]-2-methoxybenzamide,
cis-4-amino-5-chloro-N-[1-((1-(2-methylpropanethioyl)piperidin-4-yl)methyl)-3-methoxypiperidin-4-yl]-2-methoxybenzamide,
cis-4-amino-5-chloro-N-[1-((1-ethanethioylpiperidin-4-yl)methyl)-3-methoxypiperidin-4-yl]-2-methoxybenzamide,
cis-4-amino-5-chloro-N-[1-((1-propanethioylpiperidin-4-yl)methyl)-3-methoxypiperidin-4-yl]-2-methoxybenzamide,
cis-4-amino-5-chloro-N-[1-((1-pentanethioylpiperidin-4-yl)methyl)-3-methoxypiperidin-4-yl]-2-methoxybenzamide,
cis-4-amino-5-chloro-N-[1-((1-hexanethioylpiperidin-4-yl)methyl)-3-methoxypiperidin-4-yl]-2-methoxybenzamide,
cis-4-amino-5-chloro-N-[1-((1-butanethioylpiperidin-4-yl)methyl)-3-methoxypiperidin-4-yl]-2-methoxybenzamide,
cis-4-amino-5-chloro-N-[1-((1-(3-methylbutanethioyl)piperidin-4-yl)methyl)-3-methoxypiperidin-4-yl]-2-methoxybenzamide,
cis-4-amino-5-chloro-N-[1-((1-(4-methylpentanethioyl)piperidin-4-yl)methyl)-3-methoxypiperidin-4-yl]-2-methoxybenzamide,
cis-4-amino-5-chloro-N-[1-((1-(2,2-dimethylpropanethioyl)piperidin-4-yl)methyl)-3-methoxypiperidin-4-yl]-2-methoxybenzamide,
S-ethyl 4-[cis-4-(4-amino-5-chloro-2-methoxybenzamido)-3-methoxypiperidin-1-yl)methyl]piperidine-1-carbothioate,
S-propyl 4-[(cis-4-(4-amino-5-chloro-2-methoxybenzamido)-3-methoxypiperidin-1-yl)methyl]piperidine-1-carbothioate,
S-butyl 4-[cis-4-(4-amino-5-chloro-2-methoxybenzamido)-3-methoxypiperidin-1-yl)methyl]piperidine-1-carbothioate,
cis-4-amino-5-chloro-N-[1-((1-(isopropylsulfonyl)piperidin-4-yl)methyl)-3-methoxypiperidin-4-yl]-2-methoxybenzamide,
cis-4-amino-5-chloro-N-[1-((1-(methylsulfonyl)piperidin-4-yl)methyl)-3-methoxypiperidin-4-yl]-2-methoxybenzamide,
(3S,4R)-4-amino-5-chloro-N-[1-((1-(methylsulfonyl)piperidin-4-yl)methyl)-3-methoxypiperidin-4-yl]-2-methoxybenzamide,
cis-4-amino-5-chloro-N-[1 (2-(1-(methylsulfonyl)piperidin-4-yl)ethyl)-3-methoxypiperidin-4-yl]-2-methoxybenzamide,
cis-4-amino-5-chloro-N-[1-(3-(1H-1,2,4-triazol-1-yl)propyl)-3-methoxypiperidin-4-yl]-2-methoxybenzamide,
(3S,4R)-4-amino-5-chloro-N-[1-(3-(1H-1,2,4-triazol-1-yl)propyl)-3-methoxypiperidin-4-yl]-2-methoxybenzamide,
cis-4-amino-5-chloro-N-[1-(3-(1H-tetrazol-2-yl)propyl)-3-methoxypiperidin-4-yl]-2-methoxybenzamide,
cis-4-amino-5-chloro-N-[1-(3-(1H-1,2,3-triazol-1-yl)propyl)-3-methoxypiperidin-4-yl]-2-methoxybenzamide,
cis-4-amino-5-chloro-N-[1-(3-(1H-pyrrol-1-yl)propyl)-3-methoxypiperidin-4-yl]-2-methoxybenzamide,
cis-4-amino-5-chloro-N-[1-(2-(1H-pyrrol-1-yl)ethyl)-3-methoxypiperidin-4-yl]-2-methoxybenzamide,
cis-4-amino-5-chloro-N-[1-(2-(bicyclo[2.2.1]heptan-2-yl)ethyl)-3-methoxypiperidin-4-yl]-2-methoxybenzamide,
cis-4-amino-5-chloro-N-[1-(benzofuran-2-ylmethyl)-3-methoxypiperidin-4-yl]-2-methoxybenzamide,
cis-4-amino-5-chloro-N-[1-(2-(5-methyl-1,2,4-oxadiazol-3-yl)ethyl)-3-methoxypiperidin-4-yl]-2-methoxybenzamide,
(3S,4R)-4-amino-5-chloro-N-[1-(2-(5-methyl-1,2,4-oxadiazol-3-yl)ethyl)-3-methoxypiperidin-4-yl]-2-methoxybenzamide,
cis-4-amino-5-chloro-N-[1-(3-oxo-3-(quinolin-5-ylamino)propyl)-3-methoxypiperidin-4-yl]-2-methoxybenzamide,
cis-4-amino-5-chloro-N-[1-(3-oxo-3-(quinolin-6-ylamino)propyl)-3-methoxypiperidin-4-yl]-2-methoxybenzamide,
cis-4-amino-5-chloro-N-[1-(6-oxo-6-(quinolin-5-ylamino)hexyl)-3-methoxypiperidin-4-yl]-2-methoxybenzamide,
cis-4-amino-5-chloro-N-[1-(3-oxo-3-(4,6-dimethylpyridin-2-ylamino)propyl)-3-methoxypiperidin-4-yl]-2-methoxybenzamide,
cis-4-amino-5-chloro-N-[1-(6-oxo-6-(4,6-dimethylpyridin-2-ylamino)hexyl)-3-methoxypiperidin-4-yl]-2-methoxybenzamide,
cis-4-amino-5-chloro-N-[1-(3-oxo-3-(1H-indol-5-ylamino)propyl)-3-methoxypiperidin-4-yl]-2-methoxybenzamide,
cis-4-amino-5-chloro-N-[1-(6-oxo-6-(1H-indol-5-ylamino)hexyl)-3-methoxypiperidin-4-yl]-2-methoxybenzamide,
4-[cis-4-(4-amino-5-chloro-2-methoxybenzoylamino)-3-methoxy-piperidin-1-ylmethyl]-piperidine-1-carboxylic acid isopropylamide,
4-[cis-4-(4-amino-5-chloro-2-methoxybenzoylamino)-3-methoxy-piperidin-1-ylmethyl]-piperidine-1-carboxylic acid dimethylamide,
(3R,4S)-4-amino-5-chloro-N-[1-((1-isobutyrylpiperidin-4-yl)methyl)-3-methoxypiperidin-4-yl]-2-methoxybenzamide,
cis-4-amino-5-chloro-N-[1-((1-isobutyrylpiperidin-4-yl)methyl)-3-methoxypiperidin-4-yl]-2-methoxybenzamide hydrochloride,
cis-4-amino-5-chloro-N-[1-((1-isobutyrylpiperidin-4-yl)methyl)-3-methoxypiperidin-4-yl]-2-methoxybenzamide maleate,
cis-4-amino-5-chloro-N-[1-((1-pivaloylpiperidin-4-yl)methyl)-3-methoxypiperidin-4-yl]-2-methoxybenzamide hydrochloride,
cis-4-amino-5-chloro-N-[1-((1-pivaloylpiperidin-4-yl)methyl)-3-methoxypiperidin-4-yl]-2-methoxybenzamide maleate,
4-[cis-4-(4-amino-5-chloro-2-methoxybenzoylamino)-3-methoxy-piperidin-1-ylmethyl]-piperidine-1-carboxylic acid isopropylamide hydrochloride, and
4-[cis-4-(4-amino-5-chloro-2-methoxybenzoylamino)-3-methoxy-piperidin-1-ylmethyl]-piperidine-1-carboxylic acid isopropylamide maleate.

16. The composition according to claim 14, wherein the composition for the treatment of one or more disease conditions selected from the group consisting of gastroesophageal reflux disease, gastrointestinal disease, gastric motility disorder, non-ulcer dyspepsia, functional dyspepsia, irritable bowel syndrome (IBS), constipation, post-operative ileus, gastroparesis, dyspepsia, esophagitis, gastroesophageal disease, motion sickness, central nervous system disease, Alzheimer's disease, cognitive impairment, emesis, migraine, neurological disease, pain, cardiovascular disease, heart failure, cardiac arrhythmia, diabetes and apnea syndrome.

17. A method for activating a 5-HT₄ receptor, comprising administering a composition of claim 14 to a mammalian subject.

18. A use of a composition of claim 14 for activating a 5-HT₄ receptor.

19. A method for treating a disease condition mediated by 5-HT₄ receptor activity, comprising administering a compound of claim 1, or an isomer, a pharmaceutically acceptable salt or a hydrate thereof to a mammalian subject in need thereof.

20. A method for treating a disease condition, comprising administering a compound of claim 1, or an isomer, a pharmaceutically acceptable salt or a hydrate thereof to a mammalian subject in need thereof, wherein the disease condition is selected from the group consisting of gastroesophageal reflux disease, gastrointestinal disease, gastric motility disorder, non-ulcer dyspepsia, functional dyspepsia, irritable bowel syndrome (IBS), constipation, post-operative ileus, gastroparesis, dyspepsia, esophagitis, gastroesophageal disease, motion sickness, central nervous system disease, Alzheimer's disease, cognitive impairment, emesis, migraine, neurological disease, pain, cardiovascular disease, heart failure, cardiac arrhythmia, diabetes and apnea syndrome.

21. A use of a compound of claim 1, an isomer, a pharmaceutically acceptable salt or a hydrate thereof, for the preparation of a medicament for treating a disease condition mediated by 5-HT₄ receptor activity in a mammalian subject.

22. A use of a compound of claim 1, an isomer, a pharmaceutically acceptable salt or a hydrate thereof, for the preparation of a medicament for treating a disease condition in a mammalian subject, wherein the disease condition is selected from the group consisting of gastroesophageal reflux disease, gastrointestinal disease, gastric motility disorder, non-ulcer dyspepsia, functional dyspepsia, irritable bowel syndrome (IBS), constipation, post-operative ileus, gastroparesis, dyspepsia, esophagitis, gastroesophageal disease, motion sickness, central nervous system disease, Alzheimer's disease, cognitive impairment, emesis, migraine, neurological disease, pain, cardiovascular disease, heart failure, cardiac arrhythmia, diabetes and apnea syndrome.
